(19) United States
(12) Patent Application Publication
(10) Pub. No.: US 2018/0265877 A1
(43) Pub. Date: Sep. 20, 2018

(54) METHODS AND COMPOSITIONS FOR AGROBACTERIUM COMPRISING NEGATIVE SELECTION MARKERS

(71) Applicants: PIONEER HI-BRED INTERNATIONAL, INC., JOHNSTON, IA (US); E. I. DU PONT DE NEMOURS AND COMPANY, WILMINGTON, DE (US)

(72) Inventors: AJITH ANAND, WEST DES MOINES, IA (US); WILLIAM JAMES GORDON-KAMM, URBANDALE, IA (US); LOWE S KEITH, JOHNSTON, IA (US); PAMELA L. SHARPE, WILMINGTON, DE (US)

(73) Assignees: PIONEER HI-BRED INTERNATIONAL, INC., JOHNSTON, IA (US); E. I. DU PONT DE NEMOURS AND COMPANY, WILMINGTON, DE (US)

(21) Appl. No.: 15/765,026

(22) PCT Filed: Oct. 11, 2016
(86) PCT No.: PCT/US16/56375

§ 371 Date: Mar. 30, 2018

Related U.S. Application Data
(60) Provisional application No. 62/269,139, filed on Dec. 18, 2015.
(60) Provisional application No. 62/242,859, filed on Oct. 16, 2015.

(51) Int. Cl. C12N 15/65; C12N 15/82; C12N 15/74; A01H 4/00; A01H 1/04

(52) U.S. Cl. CPC C12N 15/65; C12N 15/8205; C12N 15/8209; A01H 4/00; A01H 1/04; C12N 15/743

(57) ABSTRACT

The present disclosure comprises methods and compositions comprising a plant transforming bacterium of the Order Rhizobiales comprising conditional negative selectable marker genes.

### CROSS-REFERENCE TO RELATED APPLICATIONS

This application claims the benefit of U.S. Provisional Application No. 62/242,859, filed Oct. 16, 2015 and U.S. Provisional Application No. 62/269,139, filed Dec. 18, 2015, both of which are hereby incorporated by reference.

### TECHNICAL AREA

The present disclosure comprises methods and compositions for plant transformation using bacteria of the Order Rhizobiales, including bacteria from the Genera Agrobacterium, Ochrobactrum, Ensifer, Bradyrhizobium, Rhizobium, Sinorhizobium, Phyllobacterium, or Mesorhizobium containing conditional negative selectable marker genes, for use, for example, in removing bacteria from the Genera Agrobacterium, Ochrobactrum, Ensifer, Bradyrhizobium, Rhizobium, Sinorhizobium, Phyllobacterium, or Mesorhizobium from transformed plant tissue cultures.

### REFERENCE TO A SEQUENCE LISTING SUBMITTED AS A TEXT FILE VIA EFS-WEB

The Sequence Listing submitted as a text file named “20161004_4672PCT_ST25_SeqLst.txt,” created on Oct. 4, 2016, and having a size 23 KB is hereby incorporated by reference pursuant to 37 C.F.R. § 1.52(e)(5).

### BACKGROUND

Plant transformation using bacteria of the Order Rhizobiales, including bacteria from the Genera Agrobacterium, Ochrobactrum, Ensifer, Bradyrhizobium, Rhizobium, Sinorhizobium, Phyllobacterium, or Mesorhizobium is a widely used technique for introducing exogenous nucleic acid sequences (genetic information) into plant cells. Perhaps the most widely used is Agrobacterium-mediated plant transformation. Agrobacterium is a genus of bacteria of the Order Rhizobiales that have the ability to transfer DNA sequences into the genomes of plants. A widely used species of Agrobacterium is A. tumefaciens, the causal agent of the neoplastic disease crown gall in plants. A closely related species, A. rhizogenes, induces hairy root disease and also has been used for DNA transfer to plant genomes. The ability of these bacteria to transfer DNA into plants depends on the presence of large plasmids (>100 kb) within the Agrobacterium cells. These plasmids are referred to as the Ti (Tumor inducing) or Ri (Root inducing) in A. tumefaciens and A. rhizogenes, respectively. DNA transfer from the bacterium into the plant genome involves mobilization of specific T-DNA (transfer DNA) molecules from the Ti plasmid into the host cell. The T-DNA region is delineated by 25 bp referred to as the left and right borders. Exogenous DNA sequences are incorporated into a plasmid in the Agrobacterium and are transferred into plant cells in plant tissue culture.

Other bacteria of the Order Rhizobiales that have the ability to transfer DNA sequences into the genomes of plants include bacteria from Ochrobactrum, Ensifer, Bradyrhizobium, Rhizobium, Sinorhizobium, Phyllobacterium, and Mesorhizobium. The plant cells that have been contacted by the bacteria of the Order Rhizobiales, such as Agrobacterium, Ochrobactrum, Ensifer, Bradyrhizobium, Rhizobium, Sinorhizobium, Phyllobacterium, or Mesorhizobium are allowed to grow and develop under tissue culture conditions. Because the bacteria of the Order Rhizobiales, such as Agrobacterium, Ochrobactrum, Ensifer, Bradyrhizobium, Rhizobium, Sinorhizobium, Phyllobacterium, or Mesorhizobium will also continue to proliferate under these conditions, a critical step is to eliminate the Agrobacterium, Ochrobactrum, Ensifer, Bradyrhizobium, Rhizobium, Sinorhizobium, Phyllobacterium, or Mesorhizobium cells during the plant cells' development. In current tissue culture practices, cultures containing Agrobacterium, Ochrobactrum, Ensifer, Bradyrhizobium, Rhizobium, Sinorhizobium, Phyllobacterium, or Mesorhizobium incorporate antibiotics into the plant cell tissue culture medium as a strategy to inhibit and/or kill the Agrobacterium, Ochrobactrum, Ensifer, Bradyrhizobium, Rhizobium, Sinorhizobium, Phyllobacterium, or Mesorhizobium cells. In some cases, these antibiotics may hinder plant cell tissue growth and development and additionally, adds cost to the transformation process and plant tissue development. Commonly used antibiotics include ticarcillin, cefotaxime, carbenicillin or vancomycin. Though Agrobacterium, Ochrobactrum, Ensifer, Bradyrhizobium, Rhizobium, Sinorhizobium, Phyllobacterium, or Mesorhizobium growth may be controlled or eliminated in short-term cultures, in prolonged tissue culture procedures, Agrobacterium, Ochrobactrum, Ensifer, Bradyrhizobium, Rhizobium, Sinorhizobium, Phyllobacterium, or Mesorhizobium may continue to grow and eventually overgrow the plant tissue, resulting in disposal of the entire tissue culture and containers and failure of production of the transformed plant tissue. What is needed are methods and compositions to inhibit Agrobacterium, Ochrobactrum, Ensifer, Bradyrhizobium, Rhizobium, Sinorhizobium, Phyllobacterium, or Mesorhizobium cells from the plant tissue culture conditions following Agrobacterium, Ochrobactrum, Ensifer, Bradyrhizobium, Rhizobium, Sinorhizobium, Phyllobacterium, or Mesorhizobium-mediated transfer of genetic sequences to plant cells.

### SUMMARY

The present disclosure comprises a plant transforming bacterium of the Order Rhizobiales comprising a conditional negative selectable marker gene, wherein the conditional negative selectable marker gene is codA. In an aspect, the plant transforming bacterium of the Order Rhizobiales is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales, selected from the Genera Agrobacterium, Ochrobactrum, or Ensifer. In a further aspect, the plant transforming bacterium of the Order Rhizobiales is a plant transforming bacterium from the Genus Agrobacterium. In a further aspect, the plant transforming bacterium of the Order Rhizobiales is a plant transforming bacterium from the Genus Ochrobactrum. In a further aspect, the plant transforming bacterium of the Order Rhizobiales is a plant transforming bacterium from the Genus Ensifer. In a further aspect, the plant transforming bacterium of the Order Rhizobiales is an auxotroph. In a further aspect, the auxotroph is a ThyA− auxotroph. In a further aspect, the plant transforming auxotroph bacterium is from the Genus Agrobacterium, Ochrobactrum, or Ensifer. In a further aspect, the ThyA− auxotroph bacterium is from the Genus Agrobacterium, Ochrobactrum, or Ensifer.

The present disclosure comprises a method for transferring selected nucleotide sequences to a plant, comprising, using a plant transforming bacterium of the Order Rhizobiales comprising a conditional negative selectable marker codA gene, that further comprises selected nucleotide sequences for transfer to the plant in bacterium-mediated transfer comprising co-culturing the plant transforming bacterium of the Order Rhizobiales with plant cells in tissue culture media, allowing transfer of the selected nucleotide sequences to the plant cells, and culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA-auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for removing a plant transforming bacterium of the Order Rhizobiales from plant tissue culture following bacterium-mediated nucleotide sequence transfer, comprising a conditional negative selectable marker codA gene, that further comprises selected nucleotide sequences for transfer to the plant in bacterium-mediated transfer to plant cells in plant tissue culture, and subsequent to nucleotide sequence transfer, culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA-auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for transforming a plant cell, comprising, a) co-culturing a plant cell and a plant transforming bacterium of the Order Rhizobiales comprising a conditional negative selectable marker codA gene, that further comprises selected nucleotide sequences for transfer to the plant cell in tissue culture; b) allowing transfer of the selected nucleotide sequences to the plant cell; and c) adding a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA− auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for counter selecting against a plant transforming bacteria of the Order Rhizobiales comprising a conditional negative selectable marker codA gene, comprising contacting the plant transforming bacteria of the Order Rhizobiales with a selective tissue culture medium comprising a non-toxic substrate for the conditional negative selectable marker codA gene that is converted enzymatically into a toxic compound to inhibit the growth and replication of the plant transforming bacteria of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA− auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises methods and compositions for making and using a bacterium of the Order Rhizobiales comprising in its genome, a conditional negative selectable marker gene. Methods of the present disclosure comprise transforming using a bacterium of the Order Rhizobiales, such as a bacterium selected from the Genera Bradyrhizobium, Rhizobium, Agrobacterium, Ochrobactrum, Ensifer, Sinorhizobium, Phyllobacterium, or Mesorhizobium comprising one or more selectable markers in its genome. In an aspect, a disclosed transformed bacterium of the Order Rhizobiales may comprise two selectable marker genes in its genome, wherein at least one selectable marker gene is a conditional negative selectable marker gene. Methods disclosed herein comprise use of a bacterium of the Order Rhizobiales comprising one or more selectable marker genes for transforming a plant cell and removal of the bacterium of the Order Rhizobiales from the plant cell tissue culture. Methods disclosed herein comprise a method for negative selection of a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium, comprising at least one negative selectable marker gene. Compositions disclosed herein comprise a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium comprising one or more selectable marker genes. In an aspect, a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium may comprise two selectable marker genes, wherein at least one selectable marker gene is a conditional negative selectable marker gene. Compositions disclosed herein comprise a vector comprising at least one selectable marker gene.

### DESCRIPTION OF FIGURES

FIG. 1 illustrates a DNA construct disclosed herein.

FIG. 2 illustrates protein sequences of type I toxins with predicted transmembrane domains indicated with gray shading.

### DETAILED DESCRIPTION

The following detailed description is provided to aid those skilled in the art in practicing the present disclosure, and should not be construed to unduly limit the present aspects of the invention as modifications and variations in the aspects discussed herein can be made by those of ordinary skill in the art without departing from the spirit or scope of the inventive discovery.

All publications, patents, patent applications and other references cited in this application are herein incorporated by reference in their entirety as if each publication, patent, patent application or other reference were specifically and individually indicated to be incorporated by reference in its entirety.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

The present disclosure comprises a plant transforming bacterium of the Order Rhizobiales comprising a conditional negative selectable marker gene, wherein the conditional negative selectable marker gene is codA. In an aspect, the plant transforming bacterium of the Order Rhizobiales is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales, selected from the Genera Agrobacterium, Ochrobactrum, or Ensifer. In a further aspect, the plant transforming bacterium of the Order Rhizobiales is a plant transforming bacterium from the Genus Agrobacterium. In a further aspect, the plant transforming bacterium of the Order Rhizobiales is a plant transforming bacterium from the Genus Ochrobactrum. In a further aspect, the plant transforming bacterium of the Order Rhizobiales is a plant transforming bacterium from the Genus Ensifer. In a further aspect, the plant transforming bacterium of the Order Rhizobiales is an auxotroph. In a further aspect, the auxotroph is a ThyA− auxotroph. In a further aspect, the plant transforming auxotroph bacterium is from the Genus Agrobacterium, Ochrobactrum, or Ensifer. In a further aspect, the ThyA− auxotroph bacterium is from the Genus Agrobacterium, Ochrobactrum, or Ensifer.

The present disclosure comprises a method for transferring selected nucleotide sequences to a plant, comprising, using a plant transforming bacterium of the Order Rhizobiales comprising a conditional negative selectable marker codA gene, that further comprises selected nucleotide sequences for transfer to the plant in bacterium-mediated transfer comprising co-culturing the plant transforming bacterium of the Order Rhizobiales with plant cells in tissue culture media, allowing transfer of the selected nucleotide sequences to the plant cells, and culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA-auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for removing a plant transforming bacterium of the Order Rhizobiales from plant tissue culture following bacterium-mediated nucleotide sequence transfer, comprising a conditional negative selectable marker codA gene, that further comprises selected nucleotide sequences for transfer to the plant in bacterium-mediated transfer to plant cells in plant tissue culture, and subsequent to nucleotide sequence transfer, culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA-auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for transforming a plant cell, comprising, a) co-culturing a plant cell and a plant transforming bacterium of the Order Rhizobiales comprising a conditional negative selectable marker codA gene, that further comprises selected nucleotide sequences for transfer to the plant cell in tissue culture; b) allowing transfer of the selected nucleotide sequences to the plant cell; and c) adding a selective tissue culture medium comprising a non-toxic substrate for the conditional negative selectable marker codA gene that is converted enzymatically into a toxic compound to inhibit the growth and replication of the plant transforming bacterium of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA− auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises a method for counter selecting against a plant transforming bacteria of the Order Rhizobiales comprising a conditional negative selectable marker codA gene, comprising contacting the plant transforming bacteria of the Order Rhizobiales with a selective tissue culture medium comprising a non-toxic substrate for the conditional negative selectable marker codA gene that is converted enzymatically into a toxic compound to inhibit the growth and replication of the plant transforming bacteria of the Order Rhizobiales. In an aspect, the plant transforming bacterium of the Order Rhizobiales useful in the method is selected from the Genera Bradyrhizobium, Rhizobium, Agrobacterium, Ochrobactrum, Ensifer, Sinorhizobium, Phyllobacterium, or Mesorhizobium. In a further aspect, the plant transforming bacterium of the Order Rhizobiales useful in the method, selected from the Genera Agrobacterium, Ochrobactrum, or Ensifer. In a further aspect, the plant transforming bacterium of the Order Rhizobiales useful in the method is a plant transforming bacterium from the Genus Agrobacterium. In a further aspect, the plant transforming bacterium of the Order Rhizobiales useful in the method is a plant transforming bacterium from the Genus Ochrobactrum. In a further aspect, the plant transforming bacterium of the Order Rhizobiales useful in the method is a plant transforming bacterium from the Genus Ensifer. In a further aspect, the plant transforming bacterium of the Order Rhizobiales useful in the method is an auxotroph. In a further aspect, the auxotroph useful in the method is a ThyA− auxotroph. In a further aspect, the plant transforming auxotroph bacterium useful in the method is from the Genus Agrobacterium, Ochrobactrum, or Ensifer. In a further aspect, the ThyA− auxotroph bacterium useful in the method is from the Genus Agrobacterium, Ochrobactrum, or Ensifer. In a further aspect, the selective tissue culture medium lacks the substrate necessary for an auxotroph bacterium's growth and replication.

The present disclosure comprises methods and compositions for making and using a bacterium of the Order Rhizobiales comprising in its genome, a conditional negative selectable marker gene. Methods of the present disclosure comprise transforming using a bacterium of the Order Rhizobiales, such as a bacterium selected from the Genera Bradyrhizobium, Rhizobium, Agrobacterium, Ochrobactrum, Ensifer, Sinorhizobium, Phyllobacterium, or Mesorhizobium comprising one or more selectable markers in its genome. In an aspect, a disclosed transformed bacterium of the Order Rhizobiales may comprise two selectable marker genes in its genome, wherein at least one selectable marker gene is a conditional negative selectable marker gene. Methods disclosed herein comprise use of a bacterium of the Order Rhizobiales comprising one or more selectable marker genes for transforming a plant cell and removal of the bacterium of the Order Rhizobiales from the plant cell tissue culture. Methods disclosed herein comprise a method for negative selection of a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium, comprising at least one negative selectable marker gene. Compositions disclosed herein comprise a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium comprising one or more selectable marker genes. In an aspect, a bacterium of the Order Rhizobiales, such as from the Genera Bradyrhizobium, Rhizobium, Agrobacterium, Ochrobactrum, Ensifer, Sinorhizobium, Phyllobacterium, or Mesorhizobium may comprise two selectable marker genes, wherein at least one selectable marker gene is a conditional negative selectable marker gene. Compositions disclosed herein comprise a vector comprising at least one selectable marker gene.

In an aspect, the Ochrobactrum is selected from the group consisting of Ochrobactrum haywardense H1, Ochrobactrum cytisi, Ochrobactrum daejeonense, Ochrobactrum lupine, Ochrobactrum oryzae, Ochrobactrum tritici, LBNL 124-A-10, HTG3-C-07, Ochrobactrum pectoris, Ochrobactrum ciceri, Ochrobactrum gallinifaecis, Ochrobactrum grignonense, Ochrobactrum guangzhouense, Ochrobactrum haematophilum, Ochrobactrum intermedium, Ochrobactrum lupini, Ochrobactrum oryzae, Ochrobactrum pecoris, Ochrobactrum pituitosum, Ochrobactrum pseudintermedium, Ochrobactrum pseudogrignonense, Ochrobactrum rhizosphaerae, and Ochrobactrum thiophenivorans (PCT/US2016/049135 incorporated herein by reference in its entirety).

In an aspect, the Sinorhizobium is selected from the group consisting of Sinorhizobium meliloti, Sinorhizobium freddi, Sinorhizobium meliloti SD630, Sinorhizobium meliloti USDA1002, Sinorhizobium fredii USDA205, Sinorhizobium fredii SF542G, Sinorhizobium fredii SF4404, and Sinorhizobium fredii SM542C (PCT/US2007/069053 incorporated herein by reference in its entirety and U.S. Pat. No. 7,888,552 incorporated herein by reference in its entirety).

In an aspect the Agrobacterium is selected from the group consisting of Agrobacterium tumefaciens and Agrobacterium rhizogenes (U.S. Pat. No. 7,888,552 incorporated herein by reference in its entirety).

In an aspect, the Rhizobium is selected from the group consisting of Rhizobium leguminosarum, Rhizobium leguminosarum Madison, Rhizobium leguminosarum USDA2370, Rhizobium leguminosarum USDA2408, Rhizobium leguminosarum USDA2668, Rhizobium leguminosarum 2370G, Rhizobium leguminosarum 2370LBA, Rhizobium leguminosarum 2048G, Rhizobium leguminosarum 2048LBA, Rhizobium leguminosarum bv. phaseoli, R. leguminosarum bv. phaseoli 2668G, Rhizobium leguminosarum bv. phaseoli 2668LBA, Rhizobium leguminosarum RL542C, Rhizobium leguminosarum bv. viciae, Rhizobium leguminosarum bv. trifolii, Rhizobium etli USDA 9032, Rhizobium etli bv. phaseoli, and Rhizobium tropici (U.S. Pat. No. 7,888,552 incorporated herein by reference in its entirety).

In an aspect, the Mesorhizobium is selected from the group consisting of Mesorhizobium loti, Mesorhizobium loti ML542G, Mesorhizobium loti ML4404 (U.S. Pat. No. 7,888,552 incorporated herein by reference in its entirety).

In an aspect, the Bradyrhizobium is selected from the group consisting of Bradyrhizobium biumjaponicum USDA 6, and B. japonicum USDA 110 (U.S. Pat. No. 7,888,552 incorporated herein by reference in its entirety).

The present disclosure comprises methods and compositions comprising a bacterium of the Order Rhizobiales, such as Bradyrhizobium, Rhizobium, Agrobacterium, Ochrobactrum, Ensifer, Sinorhizobium, Phyllobacterium, or Mesorhizobium, comprising at least one selectable marker, and/or a deleterious sequence or protein. In an aspect, at least one selectable marker is a negative selectable marker that is effective in inhibiting growth or killing Bradyrhizobium, Rhizobium, Agrobacterium, Ochrobactrum, Ensifer, Sinorhizobium, Phyllobacterium, or Mesorhizobium. For example, disclosed herein is Agrobacterium comprising a gene for a negative selectable marker, codA, which encodes CODA protein (SEQ ID NO: 4), a conditional selection marker that converts 5-fluorocytosine, a non-toxic compound, to 5-fluorouracil, a compound toxic for Agrobacterium.

Bradyrhizobium, Rhizobium, Agrobacterium, Ochrobactrum, Ensifer, Sinorhizobium, Phyllobacterium, or Mesorhizobium are used to insert DNA sequences into plant cells that are then grown in plant tissue culture to generate plant tissues or whole plants that comprise the inserted DNA sequences. As the plant cells are grown under tissue culture conditions, the Bradyrhizobium, Rhizobium, Agrobacterium, Ochrobactrum, Ensifer, Sinorhizobium, Phyllobacterium, or Mesorhizobium remains in the cultures along with the plants unless the growth of the Bradyrhizobium, Rhizobium, Agrobacterium, Ochrobactrum, Ensifer, Sinorhizobium, Phyllobacterium, or Mesorhizobium is inhibited or the Bradyrhizobium, Rhizobium, Agrobacterium, Ochrobactrum, Ensifer, Sinorhizobium, Phyllobacterium, or Mesorhizobium is killed. Currently, antibiotics are added to the culture media in an effort to control the growth of Bradyrhizobium, Rhizobium, Agrobacterium, Ochrobactrum, Ensifer, Sinorhizobium, Phyllobacterium, or Mesorhizobium in the culture. Certain plant tissues such as maize leaves, when stressed and/or wounded, leak metabolites that are very favorable to the growth of Agrobacterium, including the THY− mutant of Agrobacterium strain LBA4404thy− (see Ranch et al., 2012, U.S. Pat. No. 8,334,429 B2, incorporated herein by reference in its entirety), a thymidine auxotroph that grows poorly or not at all in the absence of exogenous thymidine in the medium. While growth of the bacterium can be inhibited in the early stages of the sub-culture process (i.e. one week), during prolonged tissue culture regimes required to recover transgenic maize callus (6-10 weeks), the overgrowth of Agrobacterium is exacerbated because supervirulent Agrobacterium, such as AGL0 or AGL1 (strains engineered to contain the Ti plasmid, pTiBo542, harboring additional vir genes originating from the Agrobacterium strain A281), grow vigorously on maize tissue culture medium with high levels of certain sugars or ions such as Cu++ which are necessary for optimal growth of corn leaf-derived calli. Agrobacterium overgrowth inhibits the corn cell culture response (hypothesized to be due to an enhanced cell death response and release of active free radicals) and at this juncture it becomes very difficult, if not undoable, to control bacterial overgrowth using conventional combinations of antibiotics currently used in tissue culture. Irrespective of the explant initially being transformed by Bradyrhizobium, Rhizobium, Agrobacterium, Ochrobactrum, Ensifer, Sinorhizobium, Phyllobacterium, or Mesorhizobium (for example, immature embryos, mature seed-derived embryos or leaf tissue) the majority of maize inbred cells produce a very compact, non-friable callus, which can make Bradyrhizobium, Rhizobium, Agrobacterium, Ochrobactrum, Ensifer, Sinorhizobium, Phyllobacterium, or Mesorhizobium persistence a problem which may be intractable to antibiotic selection.

An aspect of this disclosure provides methods and compositions for reducing or inhibiting Agrobacterium growth in plant tissue culture through using wild-type or auxotrophic Agrobacterium comprising 1) constitutive or inducible expression of one or more selectable markers, e.g., the codA gene, in the Agrobacterium, and/or 2) constitutive or inducible functioning of deleterious proteins or sequences in Agrobacterium. For example, in an Agrobacterium comprising a nucleic acid sequence expressing an enzyme that is a negative selectable marker, is present in culture media comprising a normally non-toxic substrate, the selectable marker enzyme will convert the non-toxic substance into an inhibitory product, thus providing a conditional selection and/or counter-selection of the Agrobacterium.

The present disclosure provides methods for transforming plant cells and making expression constructs and bacteria that are useful in the disclosed methods. The disclosure involves the insertion of a sequence encoding a protein or nucleotide sequence that, when expressed, is deleterious to the bacterium or a sequence encoding at least one selectable marker, such as a negative selectable marker, which may or may not be under control of an inducible regulatory sequence, into a bacterium of the Order Rhizobiales, such as Bradyrhizobium, Rhizobium, Agrobacterium, Ochrobactrum, Ensifer, Sinorhizobium, Phyllobacterium, or Mesorhizobium, and in particular, in Agrobacterium tumefaciens. In some aspects, the present disclosure provides for the efficient counter-selection of the bacterium without the use of antibiotic supplementation of the culture medium.

In an aspect, the disclosure provides a bacterium of the Order Rhizobiales, such as Bradyrhizobium, Rhizobium, Agrobacterium, Ochrobactrum, Ensifer, Sinorhizobium, Phyllobacterium, or Mesorhizobium, useful for the transfer of heterologous polynucleotide sequences into a host cell. The bacterium has, as part of its genome, either in the bacterial chromosome(s) or on a plasmid, a recombinant nucleic acid sequence comprising a nucleotide sequence encoding a protein, such as an enzyme that functions as a negative selectable marker. In an aspect, the nucleotide sequence may be operably linked to a regulatory sequence. In an aspect, a disclosed bacterium comprises a recombinant nucleic acid sequence comprising a nucleotide sequence that can suppress a small toxin molecule. In an aspect, the nucleotide sequence may be operably linked to a regulatory sequence. In an aspect, a disclosed bacterium comprises a recombinant nucleic acid sequence encoding a protein that is a bacterial-derived toxin protein and a nucleotide sequence encoding the anti-toxin protein. In an aspect, one or more of the nucleotide sequences may be operably linked to a regulatory sequence. In an aspect, a disclosed bacterium may be an auxotroph, for example, requiring a particular substrate for growth and reproduction. Such a substrate may be added to the medium to which the bacterium is exposed and in its presence, the bacterium grows well. In the absence of the substrate, the bacterium does not grow or is killed. In an aspect, the bacterium may be an inducible auxotroph. A nucleic acid sequence is operatively linked when it is placed into a functional relationship with another nucleic acid sequence.

As used herein, proteins or nucleotide sequences that negatively affect the growth or survival of a bacterium are referred to as deleterious proteins or sequences. Such deleterious proteins or sequences include, but are not limited to, negative selectable markers, counter-selectable markers, mutations or alterations to genes that result in auxotrophic bacteria, bacterial-derived toxin genes or proteins, antisense sequences that silence deleterious sequences, antibiotic peptides such as colicins and microcins, paired toxins and antitoxin or antidote proteins. Deleterious genes or nucleic acid sequences controlling or encoding deleterious proteins disclosed herein and such genes or peptides known to those of skill in the art may be located in the chromosome(s) of the bacterium or on plasmids or other genetic constructs within the bacterium. As used herein, the term “coding region” refers to the nucleotide sequence of a gene that is translatable into a polypeptide. Methods for producing constructs comprising such nucleotide sequences are well known in the art.

As used herein, plant cells transformable by the plant transforming bacteria of the Order Rhizobiales, such as Agrobacterium, Ochrobactrum, Ensifer, Bradyrhizobium, Rhizobium, Sinorhizobium, Phyllobacterium, and Mesorhizobium include explant material including, but not limited to a plant seed, a mature seed, a cotyledon, a leaf explant, a seedling, a stem, and roots, wherein the explant material is contacted with the bacterium of the Order Rhizobiales, such as Agrobacterium, Ochrobactrum, Ensifer, Bradyrhizobium, Rhizobium, Sinorhizobium, Phyllobacterium, or Mesorhizobium.

Regulatory sequences disclosed herein and known to those of skill in the art are contemplated by the present disclosure. For example, a regulatory sequence may be a promoter that constitutively causes a nucleotide sequence (a gene) to be replicated, transcribed or translated (to express the encoded protein), or combinations thereof, or the promoter may be inducible, for example by substrates in the medium or by other regulatory sequences. For example, a regulatory sequence can comprise an inducible promoter, which may be in combination with an operator sequence, another regulatory sequence. As used herein, the term “operator” or “operator sequence” refers to a polynucleotide sequence to which a repressor protein or nucleic acid can bind, thereby regulating the expression of the gene or nucleotide sequence that is regulated by the promoter. Any inducible promoter that is functional within bacterium of the Order Rhizobiales, such as Bradyrhizobium, Rhizobium, Agrobacterium, Ochrobactrum, Ensifer, Sinorhizobium, Phyllobacterium, or Mesorhizobium, can be used. In an aspect, a regulatory sequence is one that is functional in members of the genus Agrobacterium, and in particular A. tumefaciens. Examples of regulatory sequences include, but are not limited to, the Plac promoter and operator of E. coli, the nocR gene, which encodes for the transcriptional activator of Pi2 (noc), and in the presence of the noc1 operon which encodes for the nopaline transport system of A. tumefaciens (Von Lintig et al. (1991) Molec. Plant Microbe Interaction, 4:370-378) and the P_{BAD} promoter and araC operator of E. coli (Gallegos et al. (1997) Microbiol. Mol. Biol. Rev. 61:393-410). Other non-limiting examples of inducible promoters include those derived from the lactose, arabinose, rhamnose, and xylose promoters. Additional inducible promoters include the phage lambda lambda pR promoter/cI857 repressor system which is subject to temperature induction or tetracycline promoters.

An aspect of this disclosure provides methods and compositions for a conditional lethal gene operably linked to a promoter that provides for constitutive expression of the conditional lethal gene. The addition of a non-lethal precursor molecule to the medium undergoes conversion to a lethal product molecule by the action of the expressed conditional lethal gene.

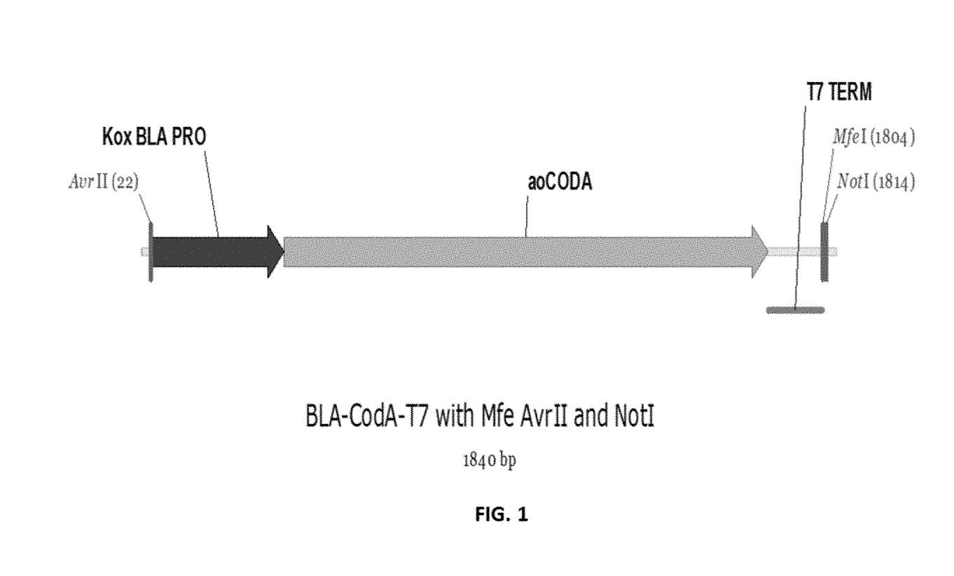

An aspect of the present disclosure provides a recombinant nucleic acid construct comprising SEQ ID NO: 1 as shown in FIG. 1. As shown in FIG. 1, the beta-lactamase promoter from Escherichia coli (BLA; SEQ ID NO: 5) was used to drive transcription of the Agrobacterium-codon-optimized codA gene (encoding the E. coli CODA protein; SEQ ID NO: 2), which was followed by a downstream E. coli T7 3′ regulatory sequence (SEQ ID NO: 6). Alternatively, the codA gene (SEQ ID NO: 3) which is not Agrobacterium-codon-optimized can be used in the construct. This nucleic acid construct is a deleterious sequence in a bacterium of the Order Rhizobiales, such as Bradyrhizobium, Rhizobium, Agrobacterium, Ochrobactrum, Ensifer, Sinorhizobium, Phyllobacterium, or Mesorhizobium, that encodes an enzyme that converts 5-fluorocytosine into 5-fluorouracil. 5-fluorocytosine is non-toxic for bacterium of the Order Rhizobiales, such as Bradyrhizobium, Rhizobium, Agrobacterium, Ochrobactrum, Ensifer, Sinorhizobium, Phyllobacterium, or Mesorhizobium, but 5-fluoruracil is toxic for such bacteria. For example, a bacterium comprising the nucleic acid construct of SEQ ID NO: 1, and expressing the gene product either constitutively or under inducible control, may be used for transferring nucleic acid sequences to plant cells in plant tissue conditions. When the bacteria is exposed to or grown in media containing 5-fluorocytosine, the gene product converts 5-fluorocytosine to 5-fluorouracil and the bacteria growth and reproduction is inhibited, for example, resulting in the bacteria being killed and removed from the tissue culture, allowing the plant tissue to continue to grow and develop without contamination by the bacteria. A composition of the present disclosure comprises a bacterium, for example, an Agrobacterium, and in particular A. tumefaciens, comprising the nucleic acid construct of SEQ ID NO: 1. A method of the present disclosure comprises using bacteria such as an Agrobacterium, and in particular A. tumefaciens, comprising the nucleic acid construct of SEQ ID NO: 1 in a method for bacterial transfer of DNA nucleic acids into at least one plant cell, growing the plant cell in media comprising the substrate for a negative selectable marker such as 5-fluorocytosine so that the gene product of the nucleic acid construct (e.g., a polynucleotide sequence expressing the CODA protein enzyme) converts the 5-fluorocytosine to 5-fluorouracil, and inhibiting the growth and reproduction of the bacteria so that the bacteria is removed from the plant tissue culture. One of skill in the art is capable of substituting other known selectable markers in the place of the gene sequence and expressed protein of SEQ ID NO: 1.

In an aspect, the present disclosure comprises a composition and method disclosed above in an auxotrophic bacteria, such as an Agrobacterium, and in particular A. tumefaciens. For example, a nucleic acid construct of SEQ ID NO: 1 or a similar nucleic acid construct expressing a negative selectable marker protein, may be inserted into DNA, such as the chromosome(s) of the bacteria, so that the bacteria is rendered auxotrophic for one or more substrates. In an aspect, the nucleic acid construct of SEQ ID NO: 1 or a similar nucleic acid construct expressing a negative selectable marker protein, may be located in the chromosome(s) of the auxotrophic bacteria or on other DNA in the auxotrophic bacteria, for example, on a plasmid, in an auxotrophic bacteria. Methods of the present disclosure comprise using an auxotrophic bacteria such as an auxotrophic Agrobacterium, and in particular an auxotrophic A. tumefaciens, that reproduces at a low rate or not at all in the absence of the substrate for which the bacteria is auxotrophic, and that comprises the nucleic acid construct of SEQ ID NO: 1, in a method for bacterial transfer of DNA nucleic acids into at least one plant cell; growing the plant cell in media lacking the substrate for which the bacteria is auxotrophic (e.g., a selective medium), and wherein the media comprises a substrate for a negative selectable marker such as 5-fluorocytosine, so that the gene product of the nucleic acid construct converts the 5-fluorocytosine to 5-fluorouracil, and inhibiting the growth and reproduction of the bacteria so that the bacteria is removed from the plant tissue culture. One of skill in the art is capable of substituting other known selectable markers in the place of the gene sequence and protein of SEQ ID NO: 1. Such use of more than one selectable marker or deleterious condition of the bacteria, such as auxotrophy, is useful in methods of multiple selection and can be used to control (inhibit growth or kill) hard-to-kill Agrobacterium strains, and or to inhibit growth or kill Agrobacterium under growth conditions where it is difficult to control Agrobacterium growth.

Nucleic acid constructs may comprise SEQ ID NO: 7, which encodes levansucrase, a negative selectable marker for bacteria.

Selectable markers that are known in the art are contemplated by the present disclosure for inclusion in methods and compositions disclosed herein. Use of negative selection for plant cells is known in the art, see Schlaman & Hooykas, 1997. Plant J 11:1377-86. (Effectiveness of codA in Arabidopsis); Gleave et al. 1999. PMB 40:223-35, (Use of CRE/loxP to excise transgenes and codA for negative selection against plants with the unexcised locus); Stougaard, 1993, Plant J 3:755-61, (Use of codA in plants as a negative marker).

Known selectable markers in plant cell selection methods include markers used outside the T-DNA borders to select against the Agrobacterium-backbone, (U.S. Pat. No. 7,575,917. Gilbertson et al.), makers that are toxic to the plant cells (US2009/0328253. Gilbertson et al.), markers used outside homologous recombination sequences (U.S. Pat. No. 5,501,967). Negative markers have been used in methods for homologous recombination in plants (US2005/0172365; US2005/0066386); in methods where the selectable marker is inside the homologous recombination sequences and an antisense sequence is outside the homologous recombination sequences (U.S. Pat. No. 5,527,674); in methods where the marker sequence is outside the homologous sequences, (Dubeau et al. 2009. Appl. Env. Micro 75:1211-4); and in methods comprising the dhlA gene from Xanthobacter which converts 1,2-dichloroethane to a toxic halogenated alcohol in Arabidopsis, (Naested et al. 1999. Plant J 18:571-576). Methods using the dao1 gene to convert D-isoleucine, developed for use in plants as either a positive or negative marker are known, (Erikson et al. 2004. Nature Biotech 22:455-58). A counter-selection method against Gram-negative bacteria, comprising inducible expression of levansucrase, to inhibit or kill Agrobacterium, is disclosed in US2002/0061579. Methods of using counter-selection markers, such as sacB, rpsL (strA), tetAR, pheS, thyA, lacY, gata-1, and ccdB, are known for bacterial genetics and pathogenesis (Reyrat, J. M., et al., Infect. Immun. (1998) 66(9):4011-4017). Methods of positive selection vectors in E. coli, Bacillus, Streptomyces, lactic acid bacteria, yeasts, and mammalian cells are known (Young-Jun, C., et al., Crit. Rev. Biotechnol. (2002) 22(3):225-244).

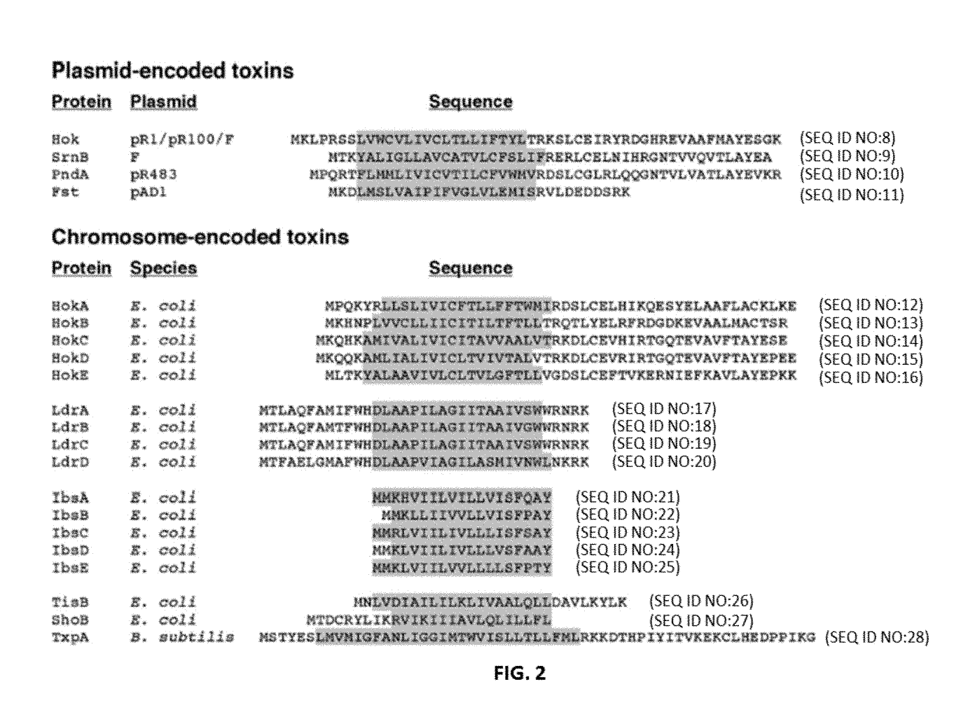

Negative selection markers enable, for example, the selection of organisms with successfully deleted sequences which encompass the marker gene (Koprek T et al. (1999) Plant J. 19(6):719-726); and negative markers may include the TK thymidine kinase (TK) and diphtheria toxin A fragment (DT-A); codA gene encoding a cytosine deaminase (Gleve A P et al. (1999) Plant Mol Biol. 40(2):223-35; Pereat R I et al. (1993) Plant Mol. Biol 23(4): 793-799; Stougaard J; (1993) Plant J 3:755-761); the cytochrome P450 gene (Koprek et al. (1999) Plant J. 16:719-726); genes encoding a haloalkane dehalogenase (Naested H (1999) Plant J. 18:571-576); the iaaH gene (Sundaresan V et al. (1995) Genes & Development 9:1797-1810); and the tms2 gene (Fedoroff N V & Smith D L 1993, Plant J. 3: 273-289). Additional suitable toxins and their polypeptide sequences (SEQ ID NOS: 8-28) are given in FIG. 2.

A negative selective marker disclosed herein is the protein CODA expressed by the gene codA, can be a conditional selective marker, in that the gene can express the CODA protein with no deleterious effect on the organism with the gene. However, when the non-toxic substrate is added to the growth medium (in this case 5-fluorocytosine), the CODA protein converts it to 5-fluorouracil, which is the toxin, and inhibits growth or kills the organism comprising the gene. In plants, codA has been used as a plant transgene for conditional negative selection. See, for example, Kobayashi, T., et al., 1995, A conditional negative selection for Arabidopsis expressing a bacterial cytosine deaminase gene, Japanese J. Genet. 70:409-422; Gallego, M., Sirnad-Pugnet, P. and C. White. 1999, Positive-negative selection and T-DNA stability in Arabidopsis transformation, Plant Mol. Boil 39(1):83-93; Kopek, T., McElroy, D., Louwerse, J., Williams-Carrier, R. and P. Lemaux, 1999, Negative selection systems for transgenic barley (Hordeum Vulgare L.): comparison of bacterial codA- and cytochrome P450 gene-mediated selection, Plant J. 16(6):719-726; Corneille, S., Lutz, K., Svab. And P. maliga, 2001, Efficient elimination of selectable marker genes from the plastid genome by the CRE-lox site-specific recombination system, Plant J. 27(2):171-178; Park, J., Lee, Y., Kang, B and W. Chung, 2004, Co-transformation using a negative selectable marker gene for the production of selectable maker gene-free transgenic plants, Theor. Appl. Genet. 109(8):1562-1567; Kondrak, M., van der Meer, I. and Z. Banfalvi, 2006, Generation of marker- and backbone-free transgenic potatoes by site-specific recombination and a bi-functional marker gene in a non-regular one-border Agrobacterium transformation vector, Transgenic Res. 15:729-737, Yan, H. and C. Rommens, 2007, Transposition-based plant transformation, Plant Physiol. 143:570-578; Dutt, M, Li Z., Dhekney S. and D. Gray, 2012, Co-transformation of grapevine somatic embryos to produce transgenic plants free of marker genes. Methods Mol. Biol. 847:201-213.

Examples of genes that can be used as negative selectable makers, whether inducible or constitutive, in bacteria include, but are not limited to, the following. A selectable marker can be a mutated version of the pheS gene, from E. coli, which encodes an alpha s-subunit of PHE-tRNA synthetase with relaxed substrate specificity. This selectable marker renders bacteria comprising it sensitive to p-chlorophenylalanine, a phenylalanine analog. The bacterium incorporates the analog into proteins, which is toxic to the bacterium. The mutation is an Ala294 to Gly294 mutation in the protein (Kast and Hennecke, 1991. Gene 44:253-263). A selectable marker for bacteria can be the Bacillus subtilis gene (sacB) that encodes the levansucrase enzyme, and its substrate is sucrose. Data indicates that this negative selectable marker is not entirely effective in that colonies can continue to grow (Bass, et al., J. Bacteriol. 1996, 178(4):1154). A less effective selectable marker, such as the sacB gene that does not completely control the growth of Agrobacterium, may be combined with one or more other deleterious proteins or sequences, or auxotrophic conditions in Agrobacterium, to provide an Agrobacterium that is effective in plant transformation and that can be removed from the tissue culture in selective media comprising the substrate for activating expression of the deleterious protein or nucleotide sequence and/or wherein the media lacks the substrate needed by the auxotroph. A less effective selectable marker is a marker that does not completely inhibit growth of a bacterium so that the bacterium is not removed from the tissue culture but continues to grow and reproduce at a low rate. Such less effective selectable markers can be combined with one or more other selectable markers and/or deleterious proteins or nucleotide sequences in normal or auxotrophic bacteria to provide bacteria that can be controlled, i.e., eliminated from a tissue culture. This control may occur without the use of traditional tissue culture antibiotics.

In an aspect, negative selectable markers used herein may be inducible or constitutive. Deleterious proteins or nucleotide sequences inserted in bacteria may be inducible. Expression of deleterious proteins in bacteria, such as Agrobacterium, include, but are not limited to, the following. Bacterial-derived toxin genes may be used for counter-selection of bacteria. For example, most bacterial plasmids have mechanisms to ensure their stable maintenance in a population of cells. These include functions such as partitioning into daughter cells and killing of cells that have lost the plasmid. These mechanisms for cell killing may be adapted for use in killing unwanted cells in a population by using substrates to induce these killing functions. One type of killing method is the secretion of antibiotic peptides such as colicins and microcins. (Pattus, F., D. Massotte, H. U. Wilmsen, J. Lakey, D. Tsernoglou, A. Tucker, and M. W. Parker. 1990. Colicins: prokaryotic killer-pores. Experientia 46:180-192. Baquero, F., and F. Moreno. 1984. The microcins. FEMS Microbiol. Lett. 23:117-175.). Cells carrying plasmids that produce a bacteriocin or a microcin are immune to the antibiotic that they produce, while cells in the population that have lost the plasmid lose their immunity to the toxin and are killed.

In an aspect, the present disclosure provides methods of controlling growth of Agrobacterium involving pairs of toxins and antitoxins or antidotes for use as negative selection markers. The antitoxin blocks the toxin activity, but may be inherently less stable than the toxin. Loss of expression of the antitoxin results in the toxin becoming active and killing the cell. See Hayes, 2003, Science 301; 1496, and Bukowski, 2011 Acta Biochim Pol. 2011; 58(1):1-9, for recent reviews. In another family of toxin systems, the toxin is regulated by antisense RNAs. See for example, Fozo, 2008, Microbiology and Molecular Biology Reviews, December 2008, p. 579-589. These toxin/antitoxin families are found in most plasmids and also in the chromosomes in most bacteria and archea. Chromosomal families include relBE, vapBC, hicAB, mazEF and phd/doc families. The E. coli chromosome contains at least 12 such loci. The activities for these toxins include ribonucleases, translational inhibitors, gyrase inhibitors, kinases and pore-forming peptides that disrupt the cytoplasmic membrane potential.

In an aspect, the present disclosure provides methods of controlling growth of bacterium of the Order Rhizobiales, such as Bradyrhizobium, Rhizobium, Agrobacterium, Ochrobactrum, Ensifer, Sinorhizobium, Phyllobacterium, or Mesorhizobium, involving expression of a toxin or its antitoxin under control of an inducible promoter or promoter for activated expression for use as negative selection markers. Induction of the promoter with the appropriate exogenous agent, e.g., a chemical inducer, allows expression of the toxin at the appropriate time and killing the cell. Suitable inducible promoters are known to one skilled in the art and are described herein.

Various promoter systems are known for activated expression of a gene of interest. For example, a gene can be operably linked to promoter that requires the presence a ligand to repress expression of the toxin or selectable marker. Examples of promoters and ligands for the activated expression of a gene of interest include the Tet-On System, i. e rtTA dependent, which requires a tetracycline derivative (such as doxycycline or anhydrotetracycline) to repress expression. In this system, removal of the tetracycline derivative activates expression of the gene of interest. In a further aspect, the IPTG-inducible (lacZ) and the arabinose-inducible (Pbad) promoters could also be used for this type of control in Agrobacterium.

Various promoter systems are known for inducible control of expression of a gene of interest. For example, a gene of interest can be operably linked to promoter that requires the presence a ligand to induce expression of the toxin or selectable marker. Examples of promoters and ligands for the activated expression of a gene of interest include the Tet-Off System, i. e tTA dependent, which requires a tetracycline derivative to induce expression. In this system, the presence of the tetracycline derivative activates expression of the gene of interest. Other inducible systems include the ethametsulfuron (“EMR”) repressor system.

Thus, in an aspect, the present disclosure provides methods of controlling growth of bacterium of the Order Rhizobiales, such as Bradyrhizobium, Rhizobium, Agrobacterium, Ochrobactrum, Ensifer, Sinorhizobium, Phyllobacterium, or Mesorhizobium, comprising pairs of toxins and antitoxins, wherein the toxin gene is operably linked to a constitutive promoter and the antitoxin gene is operably linked to an inducible promoter or a promoter for activated expression as described herein.

In an aspect, the present disclosure provides methods of controlling growth of bacterium of the Order Rhizobiales, such as Bradyrhizobium, Rhizobium, Agrobacterium, Ochrobactrum, Ensifer, Sinorhizobium, Phyllobacterium, or Mesorhizobium, comprising toxin gene that is operably linked to an inducible promoter or a promoter for activated expression as described herein.

TABLE 1
Examples of counter-selectable markers for use
in methods and compositions disclosed herein

| Counter- | |
|---|---|
| selectable | |
| marker | Mechanism of Action |
| sacB | B. subtilis gene encoding levansucrase that converts |
| | sucrose to levans that are harmful to the bacteria. |
| rpsL | Encodes the ribosomal subunit protein (S12) target |
| (strA) | of streptomycin. |
| tetAR | Confers resistance to tetracycline but sensitivity |
| | to lipophilic compounds (fusaric and quinalic acids) |
| pheS | Encodes the a subunits of Phe-tRNA synthetase, |
| | which renders bacteria sensitive to p- |
| | chlorophenylalanine, a phenyalaine analog |
| dhfr | Encodes S1 dihydrofolate reductase; confers sensitivity |
| (folA) | to trimethoprim and related compounds. |
| lacY | Encodes lactose permease, which render bacteria |
| | sensitive to t-o-nitrophenyl-b-D-galactopyranoside. |
| Gata-1 | Encodes a zinc finger DNA-binding protein which |
| | inhibits the initiation of bacterial replication. |
| ccdB | Encodes a cell-killing protein which is a potent |
| | poison of bacterial gyrase. |
| thyA- | Encodes for thymidylate synthetase protein; confers |
| | a requirement for thymine or thymidine in the media. |

TABLE 2
Toxins and Antitoxins for use in methods
and compositions disclosed herein

| Toxin | Antitoxin | |
|---|---|---|
| (amino acids) | (amino acids) | Toxin Target* |
| CcdB (101) | CcdA (72) | DNA gyrase |
| Doc (126) | Phd (73) | Translation |
| ParE (103) | ParD | DNA gyrase |
| Kid (110) | Kis (84) | DNAB |
| PasB (90) | PasA (74) | ND |
| Gamma (287) | Epsilon (90) | ND |
| HigB (92) | HigA (104) | ND |
| RelE (95) | RelB (83) | ND |
| MvpT (133) | MvpA (75) | ND |
| Txe (85) | Axe (89) | ND |
| MazF | MazE | Endoribonuclease |
| PemK | PemI | Endoribonuclease |
| ChpBk | ChpBI | Endoribonuclease |
| MazF-mt1- | MazE-mt1- | ND |
| MazF-mt7 | MazE-mt7 | |
| MazF_{SA} | MazE_{SA} | Endoribonuclease |
| PemK_{SA} | PemI_{SA} | Endoribonuclease |
| YoeB | YoeM | Endoribonuclease |
| YafO | YafM | Endoribonuclease |
| YgjN | YgjM | Endoribonuclease |
| YgiU (MqsR) | YgiT (MqsA) | Endoribonuclease |
| YafQ | DinJ | Endoribonuclease |
| VapC | VapB | Endoribonuclease |
| HipA | HipB | Ser/Thr kinase |
| HicA (YncN) | HicB (YdcQ) | Endoribonuclease |

*Suggested target; or not determined as indicated by “ND.”

In an aspect, the present disclosure provides methods of controlling growth of bacterium of the Order Rhizobiales, such as Bradyrhizobium, Rhizobium, Agrobacterium, Ochrobactrum, Ensifer, Sinorhizobium, Phyllobacterium, or Mesorhizobium, involving pairs of small toxic proteins and the antisense RNAs that repress expression of the toxic proteins. The expression of the antisense RNAs as under regulatory control that is induced by a substrate that activates or deactivates the regulatory control so that the antisense RNAs are not expressed and in the absence of the antisense RNAs, the toxic proteins are expressed and the bacterium is killed. For example, disclosed herein is a bacterium of the Order Rhizobiales, such as Bradyrhizobium, Rhizobium, Agrobacterium, Ochrobactrum, Ensifer, Sinorhizobium, Phyllobacterium, or Mesorhizobium, comprising nucleotide sequences encoding one or more small toxic proteins, such as those disclosed in FIG. 2, and the nucleotide sequences encoding one or more of the corresponding antisense RNAs that repress expression of the small toxic proteins. Methods of using such a bacterium of the Order Rhizobiales, such as Bradyrhizobium, Rhizobium, Agrobacterium, Ochrobactrum, Ensifer, Sinorhizobium, Phyllobacterium, or Mesorhizobium, for transferring selected DNA sequences into a plant cell comprise co-culturing the bacterium of the Order Rhizobiales, such as Bradyrhizobium, Rhizobium, Agrobacterium, Ochrobactrum, Ensifer, Sinorhizobium, Phyllobacterium, or Mesorhizobium, comprising nucleotide sequences encoding one or more small toxic proteins and the nucleotide sequences encoding one or more of the corresponding antisense RNAs that repress expression of the small toxic proteins, and wherein the bacterium of the Order Rhizobiales, such as Bradyrhizobium, Rhizobium, Agrobacterium, Ochrobactrum, Ensifer, Sinorhizobium, Phyllobacterium, or Mesorhizobium, also comprises selected nucleotide sequences for transfer to a plant cell, with a plant cell for a time sufficient to transfer the selected nucleotide sequences to the plant cell in tissue culture; adding to the tissue culture a selective medium comprising a substrate that induces the cessation of expression of the antisense RNAs and allows the expression of the small toxic protein so that the bacterium of the Order Rhizobiales, such as Bradyrhizobium, Rhizobium, Agrobacterium, Ochrobactrum, Ensifer, Sinorhizobium, Phyllobacterium, or Mesorhizobium, is inhibited from growing or is killed. Methods for controlling bacterium of the Order Rhizobiales, such as Bradyrhizobium, Rhizobium, Agrobacterium, Ochrobactrum, Ensifer, Sinorhizobium, Phyllobacterium, or Mesorhizobium, in tissue culture comprises providing to a tissue culture comprising a bacterium of the Order Rhizobiales, such as Bradyrhizobium, Rhizobium, Agrobacterium, Ochrobactrum, Ensifer, Sinorhizobium, Phyllobacterium, or Mesorhizobium, comprising nucleotide sequences encoding one or more small toxic proteins and the nucleotide sequences encoding one or more of the corresponding antisense RNAs that repress expression of the small toxic proteins, a selective medium comprising a substrate that induces the cessation of expression of the antisense RNAs and allows the expression of the small toxic protein so that the bacterium of the Order Rhizobiales, such as Bradyrhizobium, Rhizobium, Agrobacterium, Ochrobactrum, Ensifer, Sinorhizobium, Phyllobacterium, or Mesorhizobium, is inhibited from growing or is killed.

In various aspects, the methods of the present disclosure comprise conditional expression of an essential gene, e.g., a disclosed essential gene, under control of a regulated promoter. In a disclosed method comprising induction of the regulated promoter, the essential gene product that is required for normal growth of the bacteria is expressed in the present of an appropriate inducer. A lack of inducer in the media can be used to turn off expression of the essential gene, leading to cell death.

In various aspects, the methods of the present disclosure comprise conditional expression of an anti-sense RNA or ribozyme to target the expression of an essential gene, e.g., a disclosed essential gene. In such disclosed methods, the induction of the anti-sense RNA or ribozyme targets an essential gene transcript for inactivation, and thus can be used to block expression of the essential gene.

In various aspects, the methods of the present disclosure comprise conditional expression of bacteriophage lysis genes (for example, see Young, R., Microbiol. Rev. (1992) p. 430-481; Kalousek, et al., J. Biotechnol. (1994) 33:15-19; and Henrich, et al., Gene, (1995) 154:51-54).

In various aspects, the methods of the present disclosure comprise conditional expression of restriction endonuclease genes.

In various aspects, the methods of the present disclosure comprise engineering appropriate sensitivity to carbohydrates or other exogenous substances. For example, galactose sensitivity can be engineered in galE null cells with conditional expression of galT and galK. In the absence of gale expression, the expression of galT and galK leads to the toxic accumulation of UDP-galactose (see Ahmed, Gene (1984) 28:37-43).

Nucleic acid constructs, also referred to as expression constructs or expression cassettes, are produced using methods well known to those of ordinary skill in the art which can be found, for example, in standard texts such as Sambrook et al. Molecular Cloning, Cold Spring Harbor Laboratory Press, 1989 and Ausubel, et al. Short Protocols in Molecular Biology, Wiley & Sons, 1995. In general, constructs or expression cassettes are produced by a series of restriction enzyme digestions and ligation reactions that result in the sequences being assembled in the desired configuration. If suitable restriction sites are not available, alternative strategies, for example, the use of synthetic oligonucleotide linkers and adaptors, which are well known to those skilled in the art and described in the references cited above, can be employed to assemble the desired recombinant constructs. As is known by those of ordinary skill in the art, the precise restriction enzymes, linkers and/or adaptors required as well as the precise reaction conditions will vary with the sequences and strategies used. The assembly of recombinant constructs, however, is routine in the art and can be readily accomplished by the skilled technician without undue experimentation.

Once made, the expression constructs can be inserted into the genome of bacterium of the Order Rhizobiales, such as Bradyrhizobium, Rhizobium, Agrobacterium, Ochrobactrum, Ensifer, Sinorhizobium, Phyllobacterium, or Mesorhizobium, or introduced separately on a self-replicating plasmid of a bacterium of the Order Rhizobiales, such as Bradyrhizobium, Rhizobium, Agrobacterium, Ochrobactrum, Ensifer, Sinorhizobium, Phyllobacterium, or Mesorhizobium, used in transforming host cells. In one aspect, the bacterium of the Order Rhizobiales is Agrobacterium tumefaciens.

Any method capable of introducing the expression construct into the genome of the bacterial vector can be used. In an aspect, a construct can be inserted by the use of homologous recombination, in particular the method of Ruvkun and Ausubel ((1981) Nature, 289:85-88). In this method, a mutation, in the form of the recombinant construct of the disclosure, is directed to a specific locus on the chromosome by homologous exchange recombination. Any locus that allows the expression of the inserted nucleotide sequences can be used.

An aspect of the disclosure provides methods for transforming a plant cell using as the vector for inserting selected nucleotide sequences a bacterium of the Order Rhizobiales, such as Bradyrhizobium, Rhizobium, Agrobacterium, Ochrobactrum, Ensifer, Sinorhizobium, Phyllobacterium, or Mesorhizobium disclosed herein. In general the method involves providing a bacterium of the Order Rhizobiales, such as Bradyrhizobium, Rhizobium, Agrobacterium, Ochrobactrum, Ensifer, Sinorhizobium, Phyllobacterium, or Mesorhizobium, strain that includes a nucleotide sequence disclosed herein that expresses a deleterious protein or nucleotide sequence disclosed herein. The deleterious protein or nucleotide sequences may be operatively linked to a regulatory sequence, and the expression of the deleterious protein or nucleotide sequence may be constitutive or inducible. The nucleotide sequence(s) of interest that are to be transferred to the plant cell can be inserted within the T-DNA element of bacterium of the Order Rhizobiales, such as Bradyrhizobium, Rhizobium, Agrobacterium, Ochrobactrum, Ensifer, Sinorhizobium, Phyllobacterium, or Mesorhizobium, and introduced into the plant, for example, either directly to the resident Ti plasmid or separately using a binary plasmid strategy. Methods for the introduction of exogenous nucleotide sequences into the T-DNA element and the use of the derived bacterium of the Order Rhizobiales, such as Bradyrhizobium, Rhizobium, Agrobacterium, Ochrobactrum, Ensifer, Sinorhizobium, Phyllobacterium, or Mesorhizobium, transconjugant to transform plant cells are well known in the art (see, Maliga et al. Methods in Plant Molecular Biology, Cold Spring Harbor Laboratory Press, 1995 and U.S. Pat. No. 7,888,552 incorporated herein by reference in its entirety). The bacterium of the Order Rhizobiales, such as Bradyrhizobium, Rhizobium, Agrobacterium, Ochrobactrum, Ensifer, Sinorhizobium, Phyllobacterium, or Mesorhizobium, is subsequently used to inoculate plant cells either by direct injection or by co-cultivating the bacterium of the disclosure with individual plant cells or pieces of plants such as leaf discs. Co-cultivation of the plant and the bacterium of the Order Rhizobiales, such as Bradyrhizobium, Rhizobium, Agrobacterium, Ochrobactrum, Ensifer, Sinorhizobium, Phyllobacterium, or Mesorhizobium, is carried out in medium for a sufficient amount of time to allow the T-DNA element to be mobilized from the bacterium to the plant cell genome. Co-cultivation periods may vary for a particular plant species, but determinations are routine in the art and can be made by one of ordinary skill in the art without undue experimentation. Following co-cultivation, the transforming bacteria are counter-selected or removed from the tissue culture, for example, prior to the regeneration of the plant cells to whole plants. At this point a selective medium may be used that contains a substrate for the selectable marker, a substrate that induces a selectable marker or otherwise activates a regulatory region, and/or does not contain a substrate for an auxotrophic bacterium.

The present disclosure also includes kits comprising one or more bacteria disclosed herein. The one or more bacteria can be packaged as a component of a kit with instructions for completing the methods disclosed herein. The kits of the present disclosure can include any combination of the one or more bacteria described herein and suitable instructions (written and/or provided as audio-, visual-, or audiovisual material). In one aspect, the kit relates to a plant transformation kit for using one or more bacteria, such as bacterium of the Order Rhizobiales, such as Bradyrhizobium, Rhizobium, Agrobacterium, Ochrobactrum, Ensifer, Sinorhizobium, Phyllobacterium, or Mesorhizobium, disclosed herein. Kits utilizing any of the bacteria disclosed herein for transferring selected sequences into a plant and then controlling the growth of the bacteria are provided. For example, the kits can comprise a specific bacterium of the Order Rhizobiales, such as Bradyrhizobium, Rhizobium, Agrobacterium, Ochrobactrum, Ensifer, Sinorhizobium, Phyllobacterium, or Mesorhizobium, comprising at least one of the following: a) the bacterium of the Order Rhizobiales, such as Bradyrhizobium, Rhizobium, Agrobacterium, Ochrobactrum, Ensifer, Sinorhizobium, Phyllobacterium, or Mesorhizobium, comprises at least one selectable marker; the bacterium of the Order Rhizobiales, such as Bradyrhizobium, Rhizobium, Agrobacterium, Ochrobactrum, Ensifer, Sinorhizobium, Phyllobacterium, or Mesorhizobium, is an auxotroph; or the bacterium of the Order Rhizobiales, such as Bradyrhizobium, Rhizobium, Agrobacterium, Ochrobactrum, Ensifer, Sinorhizobium, Phyllobacterium, or Mesorhizobium, comprises a deleterious protein or nucleotide sequence having its expression under inducible regulatory control. The kits can include any reagents and materials required to carry out the methods, for example, such as substrates necessary for a selection medium.

### Definitions

The term “plant” is used herein to include any plant, tissues or organs (e.g., plant parts). Plant parts include, but are not limited to, cells, stems, roots, flowers, ovules, stamens, seeds, leaves, that can be cultured into a whole plant. A plant cell is a cell of a plant, either taken directly from a seed or plant, or derived through culture from a cell taken from a plant. Progeny, variants, and mutants of the regenerated plants are within the scope of the present disclosure, provided that these parts comprise the introduced polynucleotides.

In an aspect, plants used in methods of the present disclosure include, but are not limited to, transformation of any plant species, including, but not limited to, monocots and dicots. Examples of plants of interest include, but are not limited to, corn (Zea mays), Brassica spp. (e.g., Brassica napus, Brassica rapa, Brassica juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In the present disclosure, “nucleic acid” or “polynucleotide” refers to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

As used herein, “regulatory sequence” means a sequence of DNA concerned with controlling expression of a gene; e.g. promoters, terminators, operators and attenuators. A regulatory sequence, may, potentially operate in conjunction with the biosynthetic apparatus of a cell.

As used herein, “polynucleotide” and “oligonucleotide” are used interchangeably and mean a polymer of at least two nucleotides joined together by a phosphodiester bond and may consist of either ribonucleotides or deoxyribonucleotides.

As used herein, “sequence” means the linear order in which monomers in a polymer, for example, the order of amino acids in a polypeptide or the order of nucleotides in a polynucleotide.

As used herein, “peptide”, and “protein” are used interchangeably and mean a compound that consist of two or more amino acids that are linked by means of peptide bonds.

As used herein, “levansucrase” means a protein, a protein fragment or peptide that has the property of synthesizing a carbohydrate polymer consisting of repeating fructose residues, using sucrose as a substrate. The repeating fructose residues may be linked by beta-1 linkage or a beta-2-6 linkage or any combination of the two linkage types. The polymer of repeating fructose units may contain one terminal glucose residue, derived from a sucrose molecule, and at least two fructose residues.

As used herein, “inducer” means a substance that interacts with a regulatory sequence, either directly or indirectly, to increase the rate of transcription of the nucleotide sequence controlled by the regulatory sequence.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the present disclosure can be produced either from a nucleic acid disclosed herein, or by the use of standard molecular biology techniques.

For example, a truncated protein of the present disclosure can be produced by expression of a recombinant nucleic acid of the aspects in an appropriate host cell, or alternatively by a combination of ex vivo procedures, such as protease digestion and purification.

The term “encode” is used herein to mean that the nucleic acid comprises the required information, specified by the use of codons to direct translation of the nucleotide sequence (e.g., a legume sequence) into a specified protein. A nucleic acid encoding a protein can comprise non-translated sequences (e.g., introns) within translated regions of the nucleic acid or can lack such intervening non-translated sequences (e.g., as in cDNA).

Aspects of the disclosure encompass isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques (e.g. PCR amplification), or substantially free of chemical precursors or other chemicals when chemically synthesized. Optimally, an “isolated” polynucleotide is free of sequences (for example, protein encoding sequences) that naturally flank the polynucleotide (i.e., sequences located at the 5′ and 3′ ends of the polynucleotide) in the genomic DNA of the organism from which the polynucleotide is derived. For example, in some aspects of the disclosure, the isolated polynucleotide can contain less than about 5 kb, about 4 kb, about 3 kb, about 2 kb, about 1 kb, about 0.5 kb, or about 0.1 kb of nucleotide sequence that naturally flank the polynucleotide in genomic DNA of the cell from which the polynucleotide is derived. A protein that is substantially free of cellular material includes preparations of protein having less than about 30%, about 20%, about 10%, about 5%, or about 1% (by dry weight) of contaminating protein. When the protein of the aspects, or a biologically active portion thereof, is recombinantly produced, optimally culture medium represents less than about 30%, about 20%, about 10%, about 5%, or about 1% (by dry weight) of chemical precursors or non-protein-of-interest chemicals.

Fragments and variants relating to the nucleotide sequences and proteins encoded are within the scope of the present disclosure. A “fragment” refers to a portion of the nucleotide sequence or a portion of the amino acid sequence and thus the protein encoded therebv. Fragments of a nucleotide sequence can encode protein fragments that retain the biological activity of the native protein and have the ability to confer resistance (i.e., fungal resistance) upon a plant. Alternatively, fragments of a nucleotide sequence, that are useful as hybridization probes, do not necessarily encode fragment proteins retaining biological activity. Thus, fragments of a nucleotide sequence can range from at least about 15 nucleotides, about 50 nucleotides, about 100 nucleotides, and up to the full-length nucleotide sequence encoding the polypeptides of the present disclosure.

A fragment of a nucleotide sequence that encodes a biologically active portion of a polypeptide of the present disclosure can encode at least about 15, about 25, about 30, about 40, about 45, or about 50 contiguous amino acids, or up to the total number of amino acids present in a full-length polypeptide of the aspects disclosed. Fragments of a nucleotide sequence that are useful as hybridization probes or PCR primers generally need not encode a biologically active portion of a protein.

The term “full-length sequence,” when referring to a specified polynucleotide, means having the entire nucleic acid sequence of a native sequence. “Native sequence” is used herein to mean an endogenous sequence, i.e., a non-engineered sequence found in an organism's genome. Thus, a fragment of a nucleotide sequence of the present disclosure can encode a biologically active portion of a polypeptide, or it can be a fragment that can be used as a hybridization probe or PCR primer using methods disclosed below. A biologically active portion of a polypeptide conferring resistance can be prepared by isolating a portion of one of the nucleotide sequences of the aspects, expressing the encoded portion of the protein and assessing the ability of the encoded portion of the protein to confer or enhance fungal resistance in a plant. Nucleic acid molecules that are fragments of a nucleotide sequence of the aspects comprise at least about 15, about 20, about 50, about 75, about 100, or about 150 nucleotides, or up to the number of nucleotides present in a full-length nucleotide sequence disclosed herein.

The term “variants” means substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. One of skill in the art can recognize that variants of the nucleic acids of the aspects will be constructed such that the open reading frame is maintained. For polynucleotides, conservative variants include those sequences that, because of the degeneracy of the genetic code, encode the amino acid sequence of one of the polypeptides of the aspects. Naturally occurring allelic variants such as these can be identified with the use of well-known molecular biology techniques, as, for example, with polymerase chain reaction (PCR) and hybridization techniques as outline below. Variant polynucleotides also include synthetically derived polynucleotides, such as those generated, for example, by using site-directed mutagenesis but which still encode a protein of the aspects. Generally, variants of a particular polynucleotide of the present disclosure can have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs well known in the art.

Variants of a particular polynucleotide of the aspects (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs known in the art. Where any given pair of polynucleotides of the present disclosure is evaluated by comparison of the percent sequence identity shared by the two polypeptides they encode, wherein the percent sequence identity between the two encoded polypeptides is at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity.

“Variant protein” means a protein derived from the native protein by deletion or addition of one or more amino acids at one or more sites in the native protein and/or substitution of one or more amino acids at one or more sites in the native protein. Variant proteins, e.g., variant forms of the disclosed toxin or antitoxin polypeptides, encompassed are contemplated by the present disclosure. Variants can be prepared by site-directed or random mutagenesis methods and screened for the desired biological activity, that is, they continue to possess the desired biological activity of the native protein, which is, the ability to confer or enhance toxin or antitoxin activity as described herein. Biologically active variants of a native protein, e.g., a disclosed toxin or antitoxin polypeptide, of the aspects can have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs known in the art. A biologically active variant of a protein of the present disclosure can differ from that protein by as few as about 1-15 amino acid residues, as few as about 1-10, such as about 6-10, as few as about 5, as few as 4, 3, 2, or even 1 amino acid residue.

The proteins disclosed herein can be altered, for example, by including amino acid substitutions, deletions, truncations, and insertions. Methods for such manipulations are known in the art. For example, amino acid sequence variants and fragments of the resistance proteins can be prepared by mutations in the DNA. Methods for mutagenesis and polynucleotide alterations are known in the art.

Variant polynucleotides and proteins also encompass sequences and proteins derived from mutagenic or recombinogenic procedures, including and not limited to procedures such as DNA shuffling. Libraries of recombinant polynucleotides can be generated from a population of related sequence polynucleotides comprising sequence regions that have substantial sequence identity and can be homologously recombined in vitro or in vivo.

Sequences isolated based on their sequence identity to the entire sequences set forth herein or to variants and fragments thereof are encompassed by the present disclosure. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded protein sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species.

In a PCR approach, oligonucleotide primers can be designed for use in PCR reactions to amplify corresponding DNA sequences from cDNA or genomic DNA extracted from any organism of interest. Methods for designing PCR primers and PCR cloning are known in the art and are disclosed in Sambrook et al. (1989) Molecular Cloning: A Laboratory Manual (2d ed., Cold Spring Harbor Laboratory Press, Plainview, N.Y.). Known methods of PCR include, and are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes can be genomic DNA fragments, cDNA fragments, RNA fragments, or other oligonucleotides, and can be labeled with a detectable group such as 32P, or any other detectable marker. Thus, for example, probes for hybridization can be made by labeling synthetic oligonucleotides based on the polynucleotides of the aspects. Methods for preparation of probes for hybridization and for construction of cDNA and genomic libraries are known in the art.

Various procedures can be used to check for the presence or absence of a particular sequence of DNA, RNA, or a protein. These include, for example, Southern blots, northern blots, western blots, and ELISA analysis. These techniques are well known in the art.

The compositions and methods of the present disclosure are useful for modulating the expression levels of one or more proteins in a bacterium. The term “modulate” is used herein to mean an increase or decrease in the level of a protein within a genetically altered (i.e., transformed) bacterium relative to the level of that protein from the corresponding non-transformed bacterium (i.e., a bacterium not genetically altered in accordance with the methods of the present disclosure).

The terms “inhibit,” “inhibition,” “inhibiting”, “reduced”, “reduction” and the like as used herein to mean any decrease in the expression or function of a gene product, including any relative decrease in expression or function up to and including complete abrogation of expression or function of the gene product. The terms “inhibit,” “inhibition,” “inhibiting”, “reduced”, “reduction” and the like as used herein to mean any decrease in the growth and/or reproduction of a bacterium, up to and including complete abrogation of growth and reproduction of the bacterium, which may be referred to as death or killing the bacterium. Killing the bacteria in a tissue culture is also referred to as “controlling’ the bacteria.

The terms “increase,” “increasing,” “enhance,” “enhancing” and the like are used herein to mean any boost or gain or rise in the expression, function or activity of a gene product. Further, the terms “induce” or “increase” as used herein can mean higher expression of a gene product, such that the level is increased 10% or more, 50% or more or 100% relative to a cell lacking the gene or protein of the present disclosure.

The term “expression” as used herein in refers to the biosynthesis or process by which a polynucleotide, for example, is produced, including the transcription and/or translation of a gene product. For example, a polynucleotide of the present disclosure can be transcribed from a DNA template (such as into an mRNA or other RNA transcript) and/or the process by which a transcribed mRNA is subsequently translated into a polypeptide or protein. The term “gene product” can refer to for example, transcripts and encoded polypeptides. Inhibition of (or increase in) expression or function of a gene product (i.e., a gene product of interest) can be in the context of a comparison between any two bacteria, for example, expression or function of a gene product in a genetically altered bacterium versus the expression or function of that gene product in a corresponding wild-type bacterium. The expression level of a gene product in a wild-type bacterium can be absent.

Any method or composition that down-regulates expression of a gene product, either at the level of transcription or translation, or down-regulates functional activity of a gene product can be used to achieve inhibition of expression or function of the gene product. Similarly, any method or composition that induces or up-regulates expression of a gene product, either at the level of transcription or translation, or increases or activates or up-regulates functional activity of the gene product can be used to achieve increased expression or function of the gene or protein. Methods for inhibiting or enhancing gene expression are well known in the art.

The genes and polynucleotides of the present disclosure include naturally occurring sequences as well as mutant or altered forms, and may include variations, fragments and modified forms thereof. The proteins disclosed herein also encompass naturally occurring proteins as well as variations, fragments and modified forms thereof. Such variants and fragments will continue to possess the desired ability to be used as a selectable marker, whether conditional or not, whether negative or positive, and/or as a deleterious protein or nucleotide sequence. In an aspect, mutations made in the DNA encoding the variant or fragments thereof generally do not place the sequence out of the reading frame.

A feature of the present disclosure are methods comprising introducing a polynucleotide into a bacterium. The term “introducing” as used herein refers to presenting to the bacterium, for example, a polynucleotide. In some aspects of the present disclosure, the polynucleotide can be presented in such a manner that the sequence gains access to the interior of a cell of the plant, including its potential insertion into the genome of a plant, or may be located on a plasmid. The methods of the present disclosure do not depend on a particular method for introducing a sequence into a bacterium, only that the polynucleotide gains access to the interior of at least one bacterium. Methods for introducing polynucleotides into bacteria are known in the art.

The term “transformation” is used herein to mean the transfer of, for example, a nucleic acid fragment into the genome of a host organism, resulting in genetically stable inheritance. Host organisms containing the transformed nucleic acid fragments are referred to as “transgenic” organisms. The term “host cell” refers to the cell into which transformation of the recombinant DNA construct takes place and can include a yeast cell, a bacterial cell, and/or a plant cell. Examples of methods of plant transformation include Agrobacterium-mediated transformation that then can be used to regenerate a transformed plant by methods known to one skilled in the art.

A polynucleotide can be transiently or stably introduced into a host cell and can be maintained non-integrated, for example, as a plasmid. “Stable transformation” or “stably transformed” means that the nucleotide construct introduced into a bacterium and is capable of being inherited by the progeny thereof. “Transient transformation” as used herein means that a polynucleotide is introduced into the bacterium and does not stably reproduce so as to be found in offspring cells.

The present disclosure also comprises sequences described herein that can be provided in expression cassettes or DNA constructs for expression in the bacteria of interest. In an aspect, the cassette can include 5′ and 3′ heterologous regulatory sequences operably linked to a sequence disclosed herein. The term “operatively linked” is used herein to mean that the nucleic acid to be expressed is linked to the regulatory sequence, including promoters, terminators, enhancers and/or other expression control elements, in a manner which allows for expression of the nucleic acid. Such regulatory sequences are well known in the art and include those that direct constitutive expression of a nucleotide sequence in many types of host cells and those that direct expression of the nucleotide sequence under certain conditions. The design of the vector can depend on, for example, the type of the host cell to be transformed or the level of expression of nucleic acid desired. The cassette can contain one or more additional genes to be co-transformed into a plant. And, any additional gene(s) can be provided on multiple expression cassettes.

Expression cassettes of the present disclosure can include many restriction sites for insertion of the nucleotide sequence to be under the transcriptional regulation of the regulatory regions. The expression cassette can also contain selectable marker genes.

An expression cassette can further include in the 5′-3′ direction of transcription, a transcriptional and translational initiation region, a DNA sequence of the disclosure, and a transcriptional and translational termination region functional in bacteria. The transcriptional initiation region, the promoter, can be native or analogous or foreign or heterologous to the host cell. Additionally, the promoter can be the natural sequence or alternatively a synthetic sequence. The term “foreign” means that the transcriptional initiation region is not found in the native cell into which the transcriptional initiation region is introduced. As used herein, a chimeric gene comprises a coding sequence operably linked to a transcription initiation region that is heterologous to the coding sequence.

While it may be preferable to express the sequences using heterologous promoters, homologous promoters or native promoter sequences can be used. Such constructs may change expression levels in the host cell.

A termination region can be native with the transcriptional initiation region, native with the operably linked DNA sequence of interest, or derived from another source. Convenient termination regions are available from the Ti-plasmid of Agrobacterium tumefaciens, such as the octopine synthase and nopaline synthase termination regions.

The gene(s) can be optimized for increased expression in the transformed bacteria as needed. In other words, the genes can be synthesized using bacteria-preferred codons for improved expression. Methods for synthesizing bacteria-preferred genes are known in the art.

Additional sequence modifications are known to enhance gene expression in a cellular host. These include elimination of sequences that can be deleterious to gene expression. The G-C content of the sequence can be adjusted to levels average for a given cellular host, as calculated by reference to known genes expressed in the host cell. When possible, the sequence is modified to avoid predicted hairpin secondary mRNA structures.

The expression cassettes can additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region); potyvirus leaders, for example, TEV leader (Tobacco Etch Virus), and human immunoglobulin heavy chain binding protein (BiP); untranslated leader from the coat protein mRNA of alfalfa mosaic virus (AMV RNA 4); tobacco mosaic virus leader (TMV); and maize chlorotic mottle virus leader (MCMV) (Lommel et al. (1991) Virology 81:382 385). Other methods known to enhance translation can also be utilized, such as, introns.

The various DNA fragments can be manipulated while preparing the expression cassette, to ensure that the DNA sequences are in the proper orientation and, as appropriate, in the proper reading frame. Toward this end, adapters or linkers can be employed to join the DNA fragments. Alternatively, other manipulations can be used to provide for convenient restriction sites, removal of superfluous DNA, or removal of restriction sites. For this purpose, in vitro mutagenesis, primer repair, restriction, annealing, resubstitutions, e.g., transitions and transversions, can be involved.

Generally, the expression cassette can comprise a selectable marker gene for the selection of transformed cells. Selectable marker genes are utilized for the selection of transformed cells.

Polynucleotides described herein can be operably linked to a promoter that drives expression in a plant cell. Any promoter known in the art can be used in the methods of the present disclosure. Methods may comprise steps to express a gene from an inducible promoter, including promoters derived from regulated genes or other such regulatory sequences. Chemically-regulated promoters can be used to modulate the expression of a gene through the application of an exogenous chemical regulator. Depending upon the objective, the promoter can be a chemical-inducible promoter, where application of the chemical induces gene expression, or a chemical-repressible promoter, where application of the chemical represses gene expression.

It is to be understood that the aspects of the invention have been described in detail by way of illustration and example in order to acquaint others skilled in the art with the aspects of the invention, its principles, and practical application. Particular formulations and processes of the aspects of the invention are not limited to the descriptions of the specific aspects presented, but rather the descriptions and examples should be viewed in terms of the claims that follow and their equivalents.

It is further understood that specific aspects of the invention as set forth are not intended as being exhaustive or limiting of the aspects of the invention, and that many alternatives, modifications, and variations will be apparent to those of ordinary skill in the art in light of the foregoing examples and detailed description. Accordingly, the aspects of the invention are intended to embrace all such alternatives, modifications, and variations that fall within the spirit and scope of the following claims.

### EXAMPLES

### Example 1. Constructs for Expression of codA in Agrobacterium

The E. coli codA gene was codon-optimized for expression in Agrobacterium. The Agro-optimized gene sequence (encoding the E. coli CODA protein) was inserted between the beta-lactamase promoter (BLA PRO) sequence and the T7 terminator sequence with unique AvrII and MFeI/NotI flanking restriction sites, and this entire expression cassette was synthesized. Using the unique flanking restriction sites, the synthetic gene cassette was flanked with 5′ and 3′ sequences of the endogenous Agrobacterium AGL0 thymidylate synthase (thyA) gene. The expression cassette was then introduced into the AGL0 genome via homologous recombination disrupting the thymidylate synthase gene (creating a THY− auxotrophic mutant). See FIG. 1. One skilled in the art can introduce the codA expression cassette (BLA PRO::codA::T7 Term) into Ochrobactrum and Ensifer using 5′ and 3′ sequences from the Ochrobactrum or Ensifer thymidylate synthase gene, disrupting the thyA gene and creating a THY− auxotrophic strain with a functional codA gene. Alternatively, one skilled in the art can use a similar strategy to produce additional auxotrophic strains including, but not limited to, CYS−, LEU−, TRP−, and SER−.

### Example 2. Demonstration of Permissive Growth of Agrobacterium on Media Containing 5-Fluorocytosine, and Growth Inhibition on 5-Fluorouracil

For all Agrobacterium growth-assay experiments, standard Agrobacterium minimal medium was used, with no additives as a control treatment, or with the addition of either 5-fluorocytosine, 5-fluorouracil or thymidine. To begin these tests, experiments were conducted to test for growth of wild-type and mutant AGL0 Agrobacterium strains on either 5-fluorocytosine (5-fluorocytosine, the non-toxic substrate of the CODA protein) or 5-fluorouracil (5-fluorouracil, the toxic product). Agrobacterium cultures were grown in minimal liquid medium until reaching log phase and were then titrated to different densities (1×10⁴ or 4×10⁷ cells or Colony Forming Units (“CFU”) onto solid medium containing either basal control media (no 5-fluorocytosine or 5-fluorouracil), 500 μg/ml 5-fluorocytosine, 1000 μg/ml 5-fluorocytosine, 0.1 μg/ml 5-fluorouracil, 1 μg/ml 5-fluorouracil or 3 μg/ml 5-fluorouracil. Results are summarized in Table 3. For the four Agrobacterium strains tested (AGL0, LBA4404, EHA101 and EHA105), there was no apparent growth inhibition on the non-toxic substrate 5-fluorocytosine up to the highest concentration tested (1000 μg/ml). However, growth of all four strains was completely inhibited at very low levels of 5-fluorouracil. Comparing growth of the four strains plated at the lower density (1×10⁴ CFU) on 0.1 μg/ml of 5-fluorouracil, AGL0 and EHA105 showed the greatest growth inhibition, followed by EHA101 and LBA4404. At 1 μg/ml, all four strains plated at the lower density exhibited no growth, while growth of AGL0 originally plated at the higher density (4×10⁷ CFU) produced fewer visible colonies relative to the control treatment. At the highest concentration of 3 μg/ml 5-fluorouracil, no growth was observed for any strain. The finding that all the tested strains were totally inhibited at a 5-fluorouracil (3 μg/ml) concentration that was over 300-fold lower than the highest concentration (1000 μg/ml) of 5-fluorocytosine tested (which produced no visible growth retardation) suggests first that Agrobacterium does not have the endogenous capacity to convert the innocuous substrate 5-fluorocytosine into the toxic product 5-fluorouracil, and secondly that concentrations lower than those used in this experiment of 5-fluorocytosine in the medium can be used, as long as the enzymatic conversion by the CODA protein was reasonably efficient.

TABLE 3
Growth of different Agrobacterium strains plated
onto solid medium containing various concentrations
of either 5-fluorocytosine or 5-fluorouracil.
After 3 days, plates were scored for growth,
with a score of zero (0) indicating no visible
colonies, 1+ indicating a few isolated colonies,
progressing up to 5+ indicating a confluent lawn.

| | | 500 | 1000 | 0.1 | 1.0 | 3.0 |
|---|---|---|---|---|---|---|
| | | μg/ml | μg/ml | μg/ml | μg/ml | μg/ml |
| | | 5- | 5- | 5- | 5- | 5- |
| | 0 | fluoro- | fluoro- | fluoro- | fluoro- | fluoro- |
| Strain | Control | cytosine | cytosine | uracil | uracil | uracil |
| AGL0 | +++ | +++ | +++ | + | 0 | 0 |
| 1 × 10⁴ | | | | | | |
| AGL0 | +++++ | +++++ | +++++ | ++++ | ++++ | 0 |
| 4 × 10⁷ | | | | | | |
| LBA4404 | +++ | +++ | +++ | +++ | 0 | 0 |
| 1 × 10⁴ | | | | | | |
| EHA101 | +++ | +++ | +++ | ++ | 0 | 0 |
| 1 x 10⁴ | | | | | | |
| EHA105 | +++ | +++ | +++ | + | 0 | 0 |
| 1 × 10⁴ | | | | | | |

In Table 4, results of a more detailed kill-curve for strain AGL0 on 5-fluorouracil are shown. At plating densities ranging from 1×10³ to 1×10⁸ CFU/plate, only a few colonies grew on 0.3 μg/ml regardless of the original plating density and growth was completely inhibited at 1 μg/ml 5-fluorouracil or higher for all plating densities. This result confirmed the sensitivity of wild-type Agrobacterium strain AGL0 to very low levels of 5-fluorouracil. Finally, a third experiment was performed using either wild-type AGL0 or an auxotrophic mutant of AGL0 in which the gene encoding thymidylate synthase was inactivated, rendering the bacteria incapable of growth in the absence of thymidine (this strain was referred to as AGL0 THY−). The results are summarized in Table 5. The two strains were plated at high densities (10⁶ or 10⁸ CFU/plate) onto medium containing 0, 1 or 3 μg/ml 5-fluorouracil, with each of these three treatments containing either 0 or 50 mg/l thymidine. After 24 hours, moderate growth was observed for the wild-type AGL0 strain with no 5-fluorouracil, regardless of whether exogenous thymidine was present in the medium. In contrast, the THY− mutant showed a similar growth rate only when thymidine was present, being unable to grow in the absence of thymidine even when no 5-fluorouracil was present. At 1 and 3 μg/ml, no growth was observed for either the wild-type or THY− AGL0 strains.

One skilled in art can use a similar approach as mentioned above to test the growth inhibition of wild-type strains of Ochrobactrum or Ensifer with growth media containing varying concentrations of 5-fluorocytosine (500-100 μg/ml) and to varying concentrations of 5-flurouracil (0.1-3 μg/ml) to test the endogenous ability of the strains to covert (5-fluorocytosine to 5-fluorouracil) or the ability to grow in the media supplemented with the toxic compound 5-fluorouracil. A similar approach may be used to evaluate the THY−/coda+ strains of Ochrobactrum or Ensifer for growth in media supplemented with 5-fluorocytosine and 5-fluorouracil. When wild-type strains of Ochrobactrum or Ensifer are tested for growth on medium containing either 500 or 1000 ug/ml 5-fluorocytosine, it is expected that no growth inhibition will be observed, in contrast to being exposed to 1 or 3 μg/ml 5-fluorouracil, which is expected to prevent growth of both wild-type strains. That both strains are expected to be totally inhibited at a 5-fluorouracil concentration over 300-fold lower than the highest concentration of 5-fluorocytosine (which produces no visible growth retardation) suggests first that Ochrobactrum or Ensifer do not have the endogenous capacity to convert the innocuous substrate 5-fluorocytosine into the toxic product 5-fluorouracil, and secondly that lower concentrations of 5-fluorocytosinein the medium can be used, as long as the enzymatic conversion by the CODA protein is reasonably efficient.

TABLE 4
Growth of Agrobacterium strain AGL0 plated over a period
of 72 hours, after plating at varying densities on solid medium
containing increasing concentrations of 5-fluorouracil.

| AGL0 | 5-fluorouracil (μg/ml)* | | | | |
|---|---|---|---|---|---|
| CFU | 0 | 0.1 | 0.3 | 1 | 3 |
| 1 × 10⁸ | +++++ | +++ | + | 0 | 0 |
| 1 × 10⁷ | +++++ | ++ | + | 0 | 0 |
| 1 × 10⁶ | +++++ | ++ | + | 0 | 0 |
| 1 × 10⁵ | +++++ | +++ | + | 0 | 0 |
| 1 × 10⁴ | ++++ | ++ | + | 0 | 0 |
| 1 × 10³ | +++ | ++ | + | 0 | 0 |

*Bacterial growth was scored from no growth (0), sporadic colonies on the plate (+), increasing densities of colonies (++, +++, ++++, and finally confluent growth (+++++).

TABLE 5
Growth of Agrobacterium strain AGL0 or AGL0
THY- (scored after 24 hours) on varying
concentrations of 5-fluorouracil ± 50 mg/l thymidine.

| | 5-fluorouracil* | | 5-fluorouracil | | 5-fluorouracil | |
|---|---|---|---|---|---|---|
| | (0 μg/ml) | | (1 μg/ml) | | (3 μg/ml) | |
| | | thy | | thy | | thy |
| | 0 | (50 mg/l) | 0 | (50 mg/l) | 0 | (50 mg/l) |
| AGL0 | +++ | +++ | 0 | 0 | 0 | 0 |
| 1 x 10⁶ | | | | | | |
| AGL0 | +++ | +++ | 0 | 0 | 0 | 0 |
| 1 x 10⁸ | | | | | | |
| AGL0 | 0 | +++ | 0 | 0 | 0 | 0 |
| THY- | | | | | | |
| 1 x 10⁶ | | | | | | |
| AGL0 | 0 | +++ | 0 | 0 | 0 | 0 |
| THY- | | | | | | |
| 1 x 10⁸ | | | | | | |

*Bacterial growth was scored from no growth (0), sporadic colonies on the plate (+), increasing densities of colonies (++, +++, ++++ and finally confluent growth (+++++).

### Example 3. Growth of Agrobacterium Strain AGL0 THY−/codA+ on 5-Fluorocytosine

The newly developed AGL0 mutant strain (THY−/codA+, see Example 1) was plated on varying concentrations of 5-fluorocytosine to assess growth. This strain, along with wild-type AGL0 were cultured overnight in liquid medium, diluted to 10⁷ CFU and then 100 μl of suspension was plated onto minimal Agro medium+/− thymidine, with either 0, 100, 200 or 300 μg/ml 5-fluorocytosine, for a total of 8 treatments. After 72 hours at 28° C., the plates were scored for Agrobacterium growth.

Wild-type (non-mutated) AGL0 grew under all 5-fluorocytosine concentrations indicating that it did not produce the toxic 5-fluorouracil, and also grew in medium lacking thymidine. In the presence of 50 mg/l thymidine, the new Agrobacterium THY−/codA+ strain produced a confluent lawn of colonies after 72 hours when no 5-fluorocytosine was present. However, the THY−/codA+ strain did not grow on any concentration of 5-fluorocytosine (even though thymidine was present) even at the lowest concentration of 5-fluorocytosine tested (100 μg/ml).

In addition, the THY−/codA+ strain did not grow on medium lacking thymidine, even in the absence of 5-fluorocytosine, demonstrating that this new THY−/codA+ strain of Agrobacterium had been genetically modified to be susceptible to two different forms of counter-selection, that can now be applied simultaneously to provide more stringent elimination of Agrobacterium after plant cell transformation.

A similar strategy as described above may be used to evaluate the effectiveness of THY−/codA+ strains of Ochrobactrum or Ensifer that have been genetically modified to be susceptible to two different forms of counter-selection, and that may now be applied simultaneously to provide more stringent elimination of Ochrobactrum or Ensifer after plant cell transformation. When these newly developed THY−/coda+ strains of Ochrobactrum or Ensifer are plated onto medium containing 5-fluorocytosine, it is expected that no growth will be observed even at the lowest concentration tested (100 ug/ml). When these THY−/coda+ strains of Ochrobactrum or Ensifer are plated onto medium containing no thymidine, it is expected that growth is also prevented, demonstrating that these new THY−/codA+ strains of Ochrobactrum or Ensifer have been genetically modified to be susceptible to two different forms of counter-selection, that can be applied simultaneously to provide more stringent elimination of Ochrobactrum or Ensifer after plant cell transformation.

### Example 4. Callus Initiation from Corn Immature Embryos on Two Different Concentrations of 5-Fluorocytosine

Agrobacterium was tolerant of the substrate (5-fluorocytosine) and was inhibited by low levels of the converted product (5-fluorouracil). An experiment was performed to determine whether corn immature embryos and growing callus were sensitive to the substrate, 5-fluorocytosine. Immature embryos of two inbred lines (HC96 or PHH5G, representative non-stiff-stalk and stiff-stalk Pioneer Hi-Bred inbreds) were plated onto appropriate callus culture media (see Table 6) supplemented with 0, 100, 250 or 400 μg/ml 5-fluorocytosine and were cultured for 3 weeks. As depicted in Table 7, no discernible differences were observed in callus initiation frequency or callus growth rates after 3 weeks in culture for either inbred, indicating that callus initiation from corn immature embryos and sustained callus growth were not inhibited by these levels of 5-fluorocytosine showing that maize callus lacks the endogenous enzymatic ability to convert the non-toxic substrate 5-fluorocytosine to the toxic product 5-fluorouracil).

TABLE 6
Media composition for plant transformation and tissue culture.

| | Units | |
|---|---|---|
| Medium components | per liter | 13152C |
| MS BASAL SALT MIXTURE | g | 4.3 |
| THIAMINE•HCL | mg | 1.0 |
| L-PROLINE | G | 0.7 |
| CASEIN HYDROLYSATE (ACID) | g | 1.0 |
| MALTOSE | g | 30.0 |
| 2,4-D | mg | 1.0 |
| PHYTAGEL | g | 3.5 |
| MYO-INOSITOL | g | 0.25 |
| CUPRIC SULFATE (100 mM) | ml | 1.22 |
| AGRIBIO Carbenicillin | mg | 100 |
| BAP (1 mg/ml) | mg | 0.5 |
| pH | | 5.8 |

TABLE 7
Callus initiation frequency from immature embryos in
inbreds HC69 and PHH5G in the presence of increasing
concentrations of 5-fluorocytosine, as measured by the
percentage of embryos producing a callus response.

| | 18 d cultured on 13152C with chemical treatments | | | |
|---|---|---|---|---|
| Treatments | | Embryo with | | Embryo with |
| (5-fluorocytosine, | | culture - | | culture - |
| μg/ml) | HC69 | Response | PHH5G | Response |
| 0 | 30/50 | 60% | 8/25 | 32% |
| 100 | 32/50 | 64% | 12/50 | 24% |
| 250 | 34/50 | 68% | 18/50 | 36% |
| 400 | 34/50 | 68% | 8/25 | 32% |

### Example 5. Use of the AGL0/THY−/codA+Agrobacterium Strain for Corn Leaf Transformation

A T-DNA designed to permit recovery of transgenic events from monocot leaf tissues (shown below) was introduced into pSB1 (Komari, T., et al., Plant J. (1996) 10(1):165-174) in both the Agrobacterium strain AGL0 and the new AGL0 (THY−/codA+) strain.

When integrated into the monocot genome, this T-DNA (RB-loxP-RAB17 PRO::moCRE::pinII+ NOS PRO::ZmWUS2::pinII+UBI PRO::ZmODP2::pinII-loxP+UBI PRO::ZsGREEN::pinII::Sb-ACTIN TERM+Sb-UBI PRO::PMI::Sb-UBI TERM-LB) stimulates callus growth providing a positive selection for transgenic sectors growing out of the leaf tissue (see Gordon-Kamm et al., US20110167516). Before regeneration, CRE expression was induced excising the loxP-flanked portion of the T-DNA.

Agrobacterium-mediated transformation in leaves was performed essentially as described in Miller et al. (2002. Transgenic Research 11:381-396), with the following modifications. Maize seed was surface sterilized in a 50% Clorox, 0.05% Tween-20 solution for 20 minutes while being stirred, then, followed by three rinses with sterile distilled water. The seed were then germinated on solidified MS medium+agar for 7-14 days. At this time, the first two cm of leaf tissue above the mesocotyl were removed and diced up into roughly 1 mm fragments. These leaf fragments were then added to the Agrobacterium suspension which had been diluted to an OD of 0.6 in 10 mM MgSO4+50 mg/l thymidine+200 μM acetosyringone+0.02% Silwet® and incubated at room 25° C. for 30 minutes. The embryos were then transferred from the Agrobacterium suspension onto plates of 7101 medium+50 mg/l thymidine (see Table 8 for media composition) and co-cultured for two days at 21° C. After the two-day co-cultivation, the embryos were transferred to 605T medium (see Table 8 for media composition) for two weeks, and moved onto 13265A medium (see Table 8 for media composition) with Phytagel™ as the gelling agent. At this stage, the embryos were transferred to medium containing no thymidine and 100 μg/ml 5-fluorocytosine. The embryos were subcultured every two weeks on the same medium.

TABLE 8
Media composition for plant transformation and tissue culture.

| | Units | | | |
|---|---|---|---|---|
| | per | | | |
| Medium components | liter | 710I | 605T | 13265A |
| MS BASAL SALT MIXTURE | g | 4.3 | 4.3 | 4.3 |
| N6 MACRONUTRIENTS 10X | ml | — | 60.0 | 60.0 |
| POTASSIUM NITRATE | g | — | 1.7 | 1.7 |
| B5H MINOR SALTS 1000X | ml | — | 0.6 | 0.6 |
| NaFe EDTA FOR B5H 100X | ml | — | 6.0 | 6.0 |
| ERIKSSON'S VITAMINS | ml | — | 0.4 | 0.4 |
| 1000X (13009BASE) | | | | |
| S&H VITAMIN STOCK 100X | ml | — | 6.0 | 6.0 |
| (45BASE) | | | | |
| THIAMINE•HCL | mg | 1.0 | 0.2 | 0.5 |
| L-PROLINE | g | 0.7 | 2.0 | 2.0 |
| CASEIN HYDROLYSATE | g | — | 0.3 | 0.3 |
| (ACID) | | | | |
| SUCROSE | g | 20.0 | 20.0 | 20.0 |
| GLUCOSE | g | 10.0 | 0.6 | 10.0 |
| 2,4-D | mg | 2.0 | 0.8 | 0.8 |
| AGAR | g | 8.0 | 6.0 | — |
| PHYTAGEL | g | — | — | 3.5 |
| DICAMBA | mg | — | 1.2 | 1.2 |
| SILVER NITRATE | mg | — | 3.4 | — |
| Timentin | mg | — | 150.0 | 150 |
| Cefotaxime | mg | — | 100.0 | 100 |
| MYO-INOSITOL | g | 0.1 | — | — |
| NICOTINIC ACID | mg | 0.5 | — | — |
| PYRIDOXINE•HCL | mg | 0.5 | — | — |
| MES BUFFER | g | 0.5 | — | — |
| ACETOSYRINGONE | μM | 100.0 | — | — |
| (13017BASE) | | | | |
| ASCORBIC ACID 10 MG/ML | mg | 10.0 | — | — |
| (7S) | | | | |
| CUPRIC SULFATE (100 mM) | ml | — | — | 0.05 |
| BAP (1 mg/ml) | mg | — | — | 0.1 |
| pH | | 5.8 | 5.8 | 5.8 |

For both inbred lines (GR2HT and HN04P) used in this comparison, transgenic callus events were readily recovered using all three Agrobacterium strains (see Table 9). However, when wild-type AGL0 was used for transformation, the Agrobacterium continued to grow over this six week duration and the transgenic calli continued to become more necrotic and eventually died. Using the AGL0/THY− strain inhibited the growth of the bacteria, but the calli were still surrounded by visible bacterial growth and although the calli did not die, they had to be discarded due to contamination. Using the AGL0/THY−/codA+ strain produced good transformation results relative to the other strains (based on the number of callus events produced) with no bacterial growth. Thus, the addition of the codA conditional counter-selection system provides an effective means of eliminating Agrobacterium persistence and overgrowth of maize callus cultures.

TABLE 9
Transformation of leaf segments from two Pioneer inbred lines,
comparing the recovery of transgenic callus events and
the persistence of Agrobacteria in the culture, using either
Agrobacterium strains AGL0 (wild-type), AGL0/THY (Thy-
mutant) or AGL0/THY/codA (Thy-/codA+).

| | | Seedling | culture | | | |
|---|---|---|---|---|---|---|
| Geno- | Agro | used for | growth | # | 4 wk | 6 wk |
| type | strain | leaf Tx | rate† | events* | Agro**'† | Agro**'† |
| GR2HT | AGL0 | 30 | ++ | 44 | + | ++ |
| GR2HT | AGL0/ | 30 | ++ | 41 | −/+ | −/+ |
| | THY | | | | | |
| GR2HT | AGL0/ | 30 | ++ | 72 | − | − |
| | THY/ | | | | | |
| | CodA | | | | | |
| HN04P | AGL0 | 20 | ++ | 28 | ++ | ++ |
| HN04P | AGL0/ | 20 | ++ | 41 | −/+ | −/+ |
| | THY | | | | | |
| HN04P | AGL0/ | 20 | ++ | 21 | − | − |
| | THY/ | | | | | |
| | CodA | | | | | |

*Number of calli.
**Presence of Agrobacterium surrounding the callus.
†Bacterial growth was scored from no bacterial growth (−), minimal bacterial growth surrounding some of the plant tissue (−/+), minimal bacterial growth around all the plant tissue, or heavy bacterial growth surrounding all the plant tissue (++).

One skilled in the art may use a similar strategy as described above to control the overgrowth or persistence in the engineered THY−/coda+ strains of Ochrobactrum or Ensifer for transforming any plant explant, including but not limited to immature embryo's or leaf explants. When wild type strains of Ochrobactrum or Ensifer are used to introduce the T-DNA (RB-loxP-RAB17 PRO::moCRE::pinII+ NOS PRO::ZmWUS2::pinII+UBI PRO::ZmODP2::pinII-loxP+UBI PRO::ZsGREEN::pinII;Sb-ACTIN TERM+Sb-UBI PRO::PMI::Sb-UBI TERM-LB) into leaf tissue of Pioneer inbred HN04P, it is expected that callus events will be stimulated to grow but with continued culture the callus will become necrotic and die due to bacterial overgrowth. In contrast, when THY−/coda+ strains of Ochrobactrum or Ensifer are used to introduce the same T-DNA (RB-loxP-RAB17 PRO::moCRE::pinII+ NOS PRO::ZmWUS2::pinII+UBI PRO::ZmODP2::pinII-loxP+UBI PRO::ZsGREEN::pinII;Sb-ACTIN TERM+Sb-UBI PRO::PMI::Sb-UBI TERM-LB) into leaf tissue of Pioneer inbred HN04P, it is expected that callus events transferred onto medium containing no thymidine and 100 ug/ml 5-fluorocytosine will continue to grow with no accompanying bacterial growth.

### Example 6. Inducible Expression of a Growth-Inhibiting Gene for Agrobacterium Counter-Selection

An expression cassette is constructed containing an inducible lac promoter driving expression of the Bacillus amyloliquefaciens Barnase gene. The expression cassette is introduced into the thymidylate synthase gene of Agrobacterium strain AGL0, disrupting this endogenous gene and creating a THY−/BARNASE+ stain. This new strain is used for transformation of maize leaf tissue, and after the infection and co-cultivation periods, 1 mM IPTG is added to the medium to induce expression of BARNASE, killing the bacterium. IPTG is maintained in the maize callus subculture medium such as medium 13265+TC-agar (see Table 10 for media composition), and can even be added in later regeneration medium to insure no bacterial contamination when plants are transferred to the soil.

TABLE 10
Media composition for plant transformation and tissue culture.

| | Units | |
|---|---|---|
| Medium components | per liter | 13265 |
| MS BASAL SALT MIXTURE | g | 4.3 |
| N6 MACRONUTRIENTS 10X | ml | 60.0 |
| POTASSIUM NITRATE | g | 1.7 |
| B5H MINOR SALTS 1000X | ml | 0.6 |
| NaFe EDTA FOR B5H 100X | ml | 6.0 |
| ERIKSSON'S VITAMINS 1000X (13009BASE) | ml | 0.4 |
| S&H VITAMIN STOCK 100X (45BASE) | ml | 6.0 |
| THIAMINE•HCL | mg | 0.5 |
| L-PROLINE | g | 2.0 |
| CASEIN HYDROLYSATE (ACID) | g | 0.3 |
| SUCROSE | g | 20.0 |
| GLUCOSE | g | 10.0 |
| 2,4-D | mg | 0.8 |
| PHYTAGEL | g | 3.5 |
| DICAMBA | mg | 1.2 |
| Timentin | mg | 300 |
| Cefotaxime | mg | 100 |
| CUPRIC SULFATE (100 mM) | ml | 0.05 |
| BAP (1 mg/ml) | mg | 0.1 |
| pH | | 5.8 |

### Example 7. Plant Transformation with a Conditional Negative Selectable Marker in Ochrobactrum or Ensifer

Ochrobactrum-mediated plant transformation for the genetic improvement of plants has been demonstrated (PCT/US2016/049135 incorporated herein by reference in its entirety). Ensifer-mediated plant transformation for the genetic improvement of plants has been demonstrated (U.S. Pat. No. 9,365,858 incorporated herein by reference in its entirety).

A conditional negative selectable marker gene encoding the gene product of the codA gene may be used for controlling Ochrobactrum-overgrowth or Ensifer-overgrowth in plant cell cultures. The resultant genome modified plants may be generated with any of the following processes including Ochrobactrum-mediated or Ensifer-mediated random transformation, Ochrobactrum-mediated or Ensifer-mediated site-specific event generation, or Ochrobactrum-mediated or Ensifer-mediated genome modified event generation containing modified genes of interest in the case of genome modified event generation or containing genes of interest on a T-DNA binary vector with or without helper plasmids in the case of Ochrobactrum-mediated or Ensifer-mediated random transformation, or Ochrobactrum-mediated or Ensifer-mediated site-specific event generation. Plant material useful in these transformations and/or genome modifications may be monocot plants including, but not limited to, corn, wheat, rice, and barley, and dicot plants including, but not limited to, sunflower, Arabidopsis, safflower, soybean, alfalfa, canola, Brassica, and cotton.

1. A plant transforming bacterium of the Order Rhizobiales comprising a conditional negative selectable marker gene, wherein the conditional negative selectable marker gene is codA.

2. The plant transforming bacterium of the Order Rhizobiales of claim 1, selected from the Genera Bradyrhizobium, Rhizobium, Agrobacterium, Ochrobactrum, Ensifer, Sinorhizobium, Phyllobacterium, or Mesorhizobium.

3. The plant transforming bacterium of the Order Rhizobiales of claim 2, selected from the Genera Agrobacterium, Ochrobactrum, or Ensifer.

4. The plant transforming bacterium of the Order Rhizobiales of claim 3, wherein the plant transforming bacterium is from the Genus Agrobacterium.

5. The plant transforming bacterium of the Order Rhizobiales of claim 3, wherein the plant transforming bacterium is from the Genus Ochrobactrum.

6. The plant transforming bacterium of the Order Rhizobiales of claim 3, wherein the plant transforming bacterium is from the Genus Ensifer.

7. The plant transforming bacterium of the Order Rhizobiales of claim 4, wherein the plant transforming bacterium is an auxotroph.

8. The plant transforming bacterium of the Order Rhizobiales of claim 7, wherein the auxotroph is a ThyA− auxotroph.

9. The plant transforming bacterium of the Order Rhizobiales of claim 1, wherein the plant transforming bacterium is from the Genus Agrobacterium, Ochrobactrum, or Ensifer.

10. A method for transferring selected nucleotide sequences to a plant, comprising, using the plant transforming bacterium of the Order Rhizobiales of claim 8, that further comprises selected nucleotide sequences for transfer to the plant in bacterium-mediated transfer comprising co-culturing the plant transforming bacterium of the Order Rhizobiales with plant cells in tissue culture media, allowing transfer of the selected nucleotide sequences to the plant cells, and culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales.

11. The method of claim 10, wherein the selective tissue culture medium lacks the substrate necessary for an auxotroph bacterium's growth and replication.

12. A method for removing a plant transforming bacterium of the Order Rhizobiales from plant tissue culture following bacterium-mediated nucleotide sequence transfer, comprising, providing the plant transforming bacterium Order Rhizobiales of claim 8, that further comprises selected nucleotide sequences for transfer to the plant in bacterium-mediated transfer to plant cells in plant tissue culture, and subsequent to nucleotide sequence transfer, culturing on a selective tissue culture medium comprising a non-toxic substrate for the conditional negative selectable marker codA gene that is converted enzymatically into a toxic compound to inhibit the growth and replication of the plant transforming bacterium of the Order Rhizobiales.

13. The method of claim 12, wherein the selective tissue culture medium lacks the substrate necessary for an auxotroph bacterium's growth and replication.

14. A method for transforming a plant cell, comprising,
a) co-culturing a plant cell and the plant transforming bacterium of the Order Rhizobiales of claim 9, that further comprises selected nucleotide sequences for transfer to the plant cell in tissue culture;
b) allowing transfer of the selected nucleotide sequences to the plant cell; and
c) adding a selective tissue culture medium comprising a non-toxic substrate for the conditional negative selectable marker codA gene that is converted enzymatically into a toxic compound to inhibit the growth and replication of the plant transforming bacterium of the Order Rhizobiales.

15. The method of claim 14, wherein the selective tissue culture medium lacks the substrate necessary for an auxotroph bacterium's growth and replication.

16. A method for counter selecting against a plant transforming bacteria of the Order Rhizobiales comprising a conditional negative selectable marker gene, comprising the codA gene, comprising contacting the plant transforming bacteria of the Order Rhizobiales with a selective tissue culture medium comprising a non-toxic substrate for the conditional negative selectable marker codA gene that is converted enzymatically into a toxic compound to inhibit the growth and replication of the plant transforming bacteria of the Order Rhizobiales.

17. The method of claim 16, wherein the plant transforming bacteria of the Order Rhizobiales is selected from the Genera Bradyrhizobium, Rhizobium, Agrobacterium, Ochrobactrum, Ensifer, Sinorhizobium, Phyllobacterium, or Mesorhizobium.

18. The method of claim 17, wherein the plant transforming bacteria of the Order Rhizobiales is selected from the Genera Agrobacterium, Ochrobactrum, or Ensifer.

19. The method of claim 18, wherein the plant transforming bacteria of the Order Rhizobiales is from the Genus Agrobacterium.

20. The method of claim 18, wherein the plant transforming bacteria of the Order Rhizobiales is from the Genus Ochrobactrum.

21. The method of claim 18, wherein the plant transforming bacteria of the Order Rhizobiales is from the Genus Ensifer.

22. The method of claim 19, wherein the plant transforming bacterium of the Order Rhizobiales is an auxotroph.

23. The method claim 22, wherein the auxotroph is a ThyA− auxotroph.

24. The method of claim 16, wherein the plant transforming bacterium of the Order Rhizobiales is selected from the Genera Agrobacterium, Ochrobactrum, or Ensifer.

25. The method of claim 22, wherein the selective tissue culture medium lacks the substrate necessary for the auxotroph bacterium's growth and replication.


SEQUENCE LISTING (28 sequences)

SEQ ID NO: 1 - DNA, 1840 bp, Artificial Sequence
  Expression construct consisting of the beta-lactamase promoter from Escherichia coli; coding region for Agrobacterium-codon-optimized E. coli codA gene; and the E. coli T7 3′ terminator.
ggtctgcgttgtcgggaagacctaggggcggcgaaatcgtcggctttttgctgcgtgcgcgaggagacggcaaccacccgctggcggctgagctcttttaacgatttcacaaagcgctcggcaatccagccggggccaataatggcccaacgcagcgaggggatcgccgaggcggcgggatggcgcggttttggtaactgtgacgggaacataacggctcctgattttattttgccggggagagacactataagcaggcgcagaggaggacgaataacgaatttatgatggaattccatcatcgacatagtgcttgtcaaaagcgcgggagtcgcgtataatccgggcagctcatcactatcaaggagtctgagatgtcgaacaacgccctgcagaccatcatcaacgcccgcctgccgggcaaggaaggcctgtggcagatccatctgcaggacggcaagatctcggccatcgacgcccagtcgggcgtcatgccgatcaccgaaaactcgctggacgccgaacagggcctggtcatcccgccgttcgtcgaaccgcatatccatctggacaccacccagaccgccggccagccgaactggaaccagtcgggcaccctgttcgaaggcatcgaacgctgggccgaacgcaaggccctgctgacccatgacgacgtcaagcagcgcgcctggcagaccctgaagtggcagatcgccaacggcatccagcatgtccgcacccatgtcgacgtctcggacgccaccctgaccgccctgaaggccatgctggaagtcaagcaggaagtcgccccgtggatcgacctgcagatcgtcgccttcccgcaggaaggcatcctgtcgtatccgaacggcgaagccctgctggaagaagccctgcgcctgggcgccgacgtcgtcggcgccatcccgcatttcgaattcacccgcgaatatggcgtcgaatcgctgcataagaccttcgccctggcccagaagtatgaccgcctgatcgacgtccattgcgacgaaatcgacgacgaacagtcgcgcttcgtcgaaaccgtcgccgccctggcccatcgcgaaggcatgggcgcccgcgtcaccgcctcgcataccaccgccatgcattcgtataacggcgcctatacctcgcgcctgttccgcctgctgaagatgtcgggcatcaacttcgtcgccaacccgctggtcaacatccatctgcagggccgcttcgacacctatccgaagcgccgcggcatcacccgcgtcaaggaaatgctggaatcgggcatcaacgtctgcttcggccatgacgacgtcttcgacccgtggtatccgctgggcaccgccaacatgctgcaggtcctgcatatgggcctgcatgtctgccagctgatgggctatggccagatcaacgacggcctgaacctgatcacccatcattcggcccgcaccctgaacctgcaggactatggcatcgccgccggcaactcggccaacctgatcatcctgccggccgaaaacggcttcgacgccctgcgccgccaggtcccggtccgctattcggtccgcggcggcaaggtcatcgcctcgacccagccggcccagaccaccgtctatctggaacagccggaagccatcgactataagcgctaacggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgacaattgatagcggccgctgcgtgatctgatccttcaac

SEQ ID NO: 2 - DNA, 1284 bp, Artificial Sequence
  Agrobacterium-codon-optimized E. coli codA gene.
atgtcgaacaacgccctgcagaccatcatcaacgcccgcctgccgggcaaggaaggcctgtggcagatccatctgcaggacggcaagatctcggccatcgacgcccagtcgggcgtcatgccgatcaccgaaaactcgctggacgccgaacagggcctggtcatcccgccgttcgtcgaaccgcatatccatctggacaccacccagaccgccggccagccgaactggaaccagtcgggcaccctgttcgaaggcatcgaacgctgggccgaacgcaaggccctgctgacccatgacgacgtcaagcagcgcgcctggcagaccctgaagtggcagatcgccaacggcatccagcatgtccgcacccatgtcgacgtctcggacgccaccctgaccgccctgaaggccatgctggaagtcaagcaggaagtcgccccgtggatcgacctgcagatcgtcgccttcccgcaggaaggcatcctgtcgtatccgaacggcgaagccctgctggaagaagccctgcgcctgggcgccgacgtcgtcggcgccatcccgcatttcgaattcacccgcgaatatggcgtcgaatcgctgcataagaccttcgccctggcccagaagtatgaccgcctgatcgacgtccattgcgacgaaatcgacgacgaacagtcgcgcttcgtcgaaaccgtcgccgccctggcccatcgcgaaggcatgggcgcccgcgtcaccgcctcgcataccaccgccatgcattcgtataacggcgcctatacctcgcgcctgttccgcctgctgaagatgtcgggcatcaacttcgtcgccaacccgctggtcaacatccatctgcagggccgcttcgacacctatccgaagcgccgcggcatcacccgcgtcaaggaaatgctggaatcgggcatcaacgtctgcttcggccatgacgacgtcttcgacccgtggtatccgctgggcaccgccaacatgctgcaggtcctgcatatgggcctgcatgtctgccagctgatgggctatggccagatcaacgacggcctgaacctgatcacccatcattcggcccgcaccctgaacctgcaggactatggcatcgccgccggcaactcggccaacctgatcatcctgccggccgaaaacggcttcgacgccctgcgccgccaggtcccggtccgctattcggtccgcggcggcaaggtcatcgcctcgacccagccggcccagaccaccgtctatctggaacagccggaagccatcgactataagcgctaa

SEQ ID NO: 3 - DNA, 1284 bp, Artificial sequence
  Coding region from the E. coli codA gene.
atgtcgaataacgctttacaaacaattattaacgcccggttaccaggcaaagaggggctgtggcagattcatctgcaggacggaaaaatcagcgccattgatgcgcaatccggcgtgatgcccataactgaaaacagcctggatgccgaacaaggtttagttataccgccgtttgtggagccgcatattcacctggacaccacgcaaaccgccggacaaccgaactggaatcagtccggcacgctgtttgaaggcattgaacgctgggccgagcgcaaagcgttattaacccatgacgatgtgaaacaacgcgcctggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaattgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccgtgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagtgccagaacattgaatttgcaggattacggcattgccgccggaaacagcgcaaacctgattatcctgccggctgaaaatggatttgatgcgctgcgccgtcaggttccggtacgttattcggtacgtggcggcaaggtgattgccagtacacaaccggcacaaaccaccgtatatctggagcagccagaagccatcgattacaaacggtaa

SEQ ID NO: 4 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 5 - DNA, 348 bp, Escherichia coli
ggcggcgaaatcgtcggctttttgctgcgtgcgcgaggagacggcaaccacccgctggcggctgagctcttttaacgatttcacaaagcgctcggcaatccagccggggccaataatggcccaacgcagcgaggggatcgccgaggcggcgggatggcgcggttttggtaactgtgacgggaacataacggctcctgattttattttgccggggagagacactataagcaggcgcagaggaggacgaataacgaatttatgatggaattccatcatcgacatagtgcttgtcaaaagcgcgggagtcgcgtataatccgggcagctcatcactatcaaggagtctgag

SEQ ID NO: 6 - DNA, 144 bp, Escherichia coli
cggctgctaacaaagcccgaaaggaagctgagttggctgctgccaccgctgagcaataactagcataaccccttggggcctctaaacgggtcttgaggggttttttgctgaaaggaggaactatatccggatgatcgggcgcga

SEQ ID NO: 7 - protein, 473 aa, Bacillus subtilis
MNIKKFAKQATVLTFTTALLAGGATQAFAKETNQKPYKETYGISHITRHDMLQIPEQQKNEKYQVPEFDSSTIKNISSAKGLDVWDSWPLQNADGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASDSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 8 - protein, 52 aa, Escherichia coli
MKLPRSSLVWCVLIVCLTLLIFTYLTRKSLCEIRYRCGHREVAAFMAYESGK

SEQ ID NO: 9 - protein, 47 aa, Escherichia coli
MTKYALIGLLAVCATVLCFSLIFRERLCELNIHRGNTVVQVTLAYEA

SEQ ID NO: 10 - protein, 48 aa, Escherichia coli
MPQRTFLMMLIVICVTILCFVWMVSLCGLRLQQGNTVLVATLAYEVKR

SEQ ID NO: 11 - protein, 32 aa, Enterococcus faecalis
MKDLMSLVAIPIFVGLVLEMISRVLDEDDSRK

SEQ ID NO: 12 - protein, 48 aa, Escherichia coli
MPQKYRLLSLIVICFTLLPFTWMRDSLCLHIKQBSYBLAAFLACKLKE

SEQ ID NO: 13 - protein, 49 aa, Escherichia coli
MKHNPLVVCLLIICITILTFTLLTRQTLYELRFRDGDKEVAALMACTSR

SEQ ID NO: 14 - protein, 50 aa, Escherichia coli
MKQHKAMIVALIVICITAVVAALVTRKDLCEVHIRTGQTEVAVFTAYESE

SEQ ID NO: 15 - protein, 51 aa, Escherichia coli
MKQQKAMLIALIVICLTVIVTALVTRKDLCEVRIRTGQTEVAVFTAYEPEE

SEQ ID NO: 16 - protein, 50 aa, Escherichia coli
MLTKYALAAVIVLCLTVLGFTLLVGDSLCEFTVKERNIEFKAVLAYEPKK

SEQ ID NO: 17 - protein, 35 aa, Escherichia coli
MTLAQFAMIFWHDLAAPILAGIITAAIVSWWRNRK

SEQ ID NO: 18 - protein, 35 aa, Escherichia coli
MTLAQFAMTFWHDLAAPILAGIITAAIVGWWRNRK

SEQ ID NO: 19 - protein, 35 aa, Escherichia coli; identical to SEQ ID NO: 17

SEQ ID NO: 20 - protein, 35 aa, Escherichia coli
MTFAELGMAFWHDLAAPVIAGILASMIVNWLNKRK

SEQ ID NO: 21 - protein, 19 aa, Escherichia coli
MMKHVIILVILLVISFQAY

SEQ ID NO: 22 - protein, 18 aa, Escherichia coli
MMKLLIIVVLLVISFPAY

SEQ ID NO: 23 - protein, 19 aa, Escherichia coli
MMRLVIILIVLLLISFSAY

SEQ ID NO: 24 - protein, 19 aa, Escherichia coli
MMKLVIILIVLLLVSFAAY

SEQ ID NO: 25 - protein, 19 aa, Escherichia coli
MMKLVIILVVLLLLSFPTY

SEQ ID NO: 26 - protein, 29 aa, Escherichia coli
MNLVDIAILILKLIVAALQLLDAVLKYLK

SEQ ID NO: 27 - protein, 26 aa, Escherichia coli
MTDCRYLIKRVIKIIIAVLQLILLFL

SEQ ID NO: 28 - protein, 59 aa, Bacillus subtilis
MSTYESLMVMIGFANLIGGIMTWVISLLTLLFMLRKKDTHPIYITVKEKCLHEDPPIKG